(19) United States
(12) Patent Application Publication
(10) Pub. No.: US 2025/0186583 A1
(43) Pub. Date: Jun. 12, 2025

(54) FUSION PROTEIN OF CD137 ANTIBODY AND CD40L AND USE THEREOF

(71) Applicant: BEIJING IMMUNOAH PHARMATECH CO., LTD., Beijing (CN)

(72) Inventors: Yujie LIU, Beijing (CN); Xin GAO, Beijing (CN); Niliang QIAN, Beijing (CN); Guili XU, Beijing (CN); Jingjing WANG; Hongjie LI, Beijing (CN); Cuima YANG, Beijing (CN); Liting CHEN, Beijing (CN)

(21) Appl. No.: 18/838,155

(22) PCT Filed: Feb. 15, 2023
(86) PCT No.: PCT/CN2023/076114

§ 371 Date: Aug. 13, 2024

(30) Foreign Application Priority Data
Feb. 16, 2022 (CN) 202210140038.3

(51) Int. Cl. A61K 39/395; A61K 38/17; A61P 35/00; C07K 14/705; C07K 16/28

(52) U.S. Cl. CPC A61K 39/39558; A61K 38/1793; A61P 35/00; C07K 14/70575; C07K 16/2878; C07K 2317/55; C07K 2317/92; C07K 2319/00

(57) ABSTRACT

The present application provides a fusion protein, a method for preparing such fusion proteins and use thereof. The fusion protein comprises a Fab fragment capable of binding specifically to 4-lBB molecules and comprises CD40L capable of binding specifically to CD40 molecules; the N-terminus of the first CD40L is connected to the C-terminus of the light chain or heavy chain of the Fab fragment by means of a first peptide linker. The fusion protein of the CD137 antibody and CD40L of the present application is capable of binding specifically to CD40, has the effects of inducing dendritic cell maturation, activating lymphocytes, etc., and can be used for treating tumors and immune-related diseases; Meanwhile, the fusion protein of the CD137 antibody and CD40L can target 4-lBB and activate the signal transduction pathway of 4-lBB to enhance immune responses.

### TECHNICAL FIELD

The present application relates to the field of immunology. More specifically, the present application relates to a fusion protein of CD137 antibody and CD40L and use thereof.

### BACKGROUND OF INVENTION

CD40 is a member of the TNF receptor (TNFR) superfamily, and is mainly expressed on B cells and other antigen presenting cells (APCs), such as dendritic cells (DCs) and macrophages. CD40 ligands (CD40L) are mainly expressed on activated T cells. The interaction of CD40 and CD40L is a co-stimulatory signal of T cell activation. CD40-CD40L interaction on resting B cells can induce B cell proliferation, immunoglobulin class switching and antibody secretion, and has an effect on germinal center development and memory B cell survival, which are essential for humoral immune responses (Kehry M R., J Immunol 1996; 156: 2345-2348). Binding of CD40 to CD40L on dendritic cells can induce DC maturation, which is manifested in increased expression of costimulatory molecules, such as the B7 family (CD80 and CD86), and production of pro-inflammatory cytokines, such as interleukin 12, resulting in significant T cell responses (Stout R D et al., J Immunol 1996; 17:487-492; Brendan O'Sullivan et al., Critical Reviews in Immunology 2003; 23:83-97; Cella M et al., J Exp Med 1996; 184:747-452).

CD137 (also known as 4-1BB) is also a member of the TNFR family, and is a costimulatory molecule on CD8+ and CD4+ T cells, regulatory T cells (Tregs), natural killer T cells (NK (T) cells), B cells, and neutrophils. CD137 is not constitutively expressed on T cells, but is induced to express after activation of the T cell receptors (TCRs), for example, on tumor infiltrating lymphocytes (TILs) (Gros et al., J. Clin Invest 2014; 124(5):2246-59)).

Some CD137 stimulating antibodies have been disclosed in the prior art, including urelumab, a human IgG4 antibody (AU2004279877) and utomilumab, a human IgG2 antibody (Fisher et al., 2012 Cancer Immunol. Immunother. 61:1721-1733).

Westwood J A et al., Leukemia Research 38(2014), 948-954 discloses combination anti-CD137 and anti CD40 antibody therapy in murine myc-driven hematological cancers.

US20090074711 discloses human therapies using chimeric agonistic anti-human CD40 antibody.

Nevertheless, there remains a need in the art for fusion proteins that can target both CD40 and CD137, so as to activate cells expressing these molecules for more targeted immune induction.

### SUMMARY OF THE INVENTION

To solve the above technical problem, the present application provides a fusion protein of a novel CD137 antibody that specifically binds to CD40 and 4-1BB and CD40L. Specifically, the present application provides the following technical solutions.

In a first aspect, the present application provides a fusion protein comprising:
a) a Fab fragment specifically binding to 4-lBB molecules;
b) a first CD40L specifically binding to CD40 molecules, the N-terminus of the first CD40L being linked to the C-terminus of the light chain or heavy chain of the Fab fragment by a first peptide linker.

In some embodiments, the fusion protein further comprises:
c) a second CD40L specifically binding to CD40 molecules, the N-terminus of the second CD40L being linked to the C-terminus of the heavy chain or light chain of the Fab fragment by a second peptide linker.

In some embodiments, only one disulfide bond can be formed between the first peptide linker and the second peptide linker, and each of the peptide linkers is independently selected from the group consisting of a peptide linker comprising any of the sequences as set forth in SEQ ID NOs: 24-25, wherein X represents any amino acid other than Cys, or is absent.

In some embodiments, the first peptide linker and/or the second peptide linker can be a hinge region of a native antibody, and wherein a mutation that retains only one cysteine can be made to the hinge region.

In some embodiments, the first peptide linker and/or the second peptide linker can be the IgG1 hinge region with C239 being deleted, or the IgG1 hinge region with C239 being deleted and the hinge region D234-S252 being inverted.

In some embodiments, the fusion protein further comprises:
d) a third CD40L specifically binding to CD40 molecules, the N-terminus of the third CD40L being linked to the C-terminus of the first CD40L or the second CD40L by a third peptide linker.

In some embodiments, the third peptide linker comprises the sequence as set forth in SEQ ID NO: 5 and/or SEQ ID NO:6.

In some embodiments, the fusion protein further comprises:
e) FcBP, wherein the FcBP is linked to any one or more of the first CD40L, the second CD40L, and the third CD40L.

Preferably, the FcBP comprises or consists of the sequence as set forth in SEQ ID NO: 15.

In some embodiments, the 4-1BB molecule and the CD40 molecule are independently derived from a mammal, preferably a non-human primate or human.

In some embodiments, the Fab fragment comprises HCDR1 as set forth in SEQ ID NO: 7, HCDR2 as set forth in SEQ ID NO: 8, and HCDR3 as set forth in SEQ ID NO: 9.

In some embodiments, the Fab fragment comprises the heavy chain variable region as set forth in SEQ ID NO: 13.

In some embodiments, the Fab fragment comprises LCDR1 as set forth in SEQ ID NO: 10, LCDR2 as set forth in SEQ ID NO: 11, and LCDR3 as set forth in SEQ ID NO: 12.

In some embodiments, the Fab fragment comprises the light chain variable region as set forth in SEQ ID NO: 14.

In some embodiments, the first CD40L, the second CD40L, and the third CD40L each independently comprise any of SEQ ID NOs: 1-4.

In some embodiments, the fusion protein specifically binds to the CD40 molecule with an affinity of at least 1×10⁻⁸.

In some embodiments, the fusion protein has the function of a CD40 agonist and can induce dendritic cell maturation and/or T cell activation.

In some embodiments, the fusion protein specifically binds to the 4-1BB molecule with an affinity of at least 1×10⁻⁸.

In some embodiments, the fusion protein has the function of a 4-1BB agonist and can induce T cell activation.

In a second aspect, the present application provides a nucleic acid encoding the fusion protein of the first aspect.

In a third aspect, the present application provides an expression vector comprising the nucleic acid of the second aspect.

In a fourth aspect, the present application provides a host cell comprising the nucleic acid of the second aspect or the expression vector of the third aspect.

In a fifth aspect, the present application provides a method of preparing the fusion protein of the first aspect, comprising:
a) culturing the host cell of the fourth aspect; and
b) recovering the fusion protein from the host cells or the supernatant of the culture thereof.

In a sixth aspect, the present application provides a pharmaceutical composition comprising the fusion protein of the first aspect, the nucleic acid of the second aspect, the expression vector of the third aspect, or the host cell of the fourth aspect, and a pharmaceutically acceptable carrier.

In a seventh aspect, the present application provides use of the fusion protein of the first aspect, the nucleic acid of the second aspect, the expression vector of the third aspect, or the host cell of the fourth aspect in the manufacture of a medicament for treating, ameliorating, or preventing a tumor, an immune-related disease, or an infectious disease.

In an eighth aspect, the present application provides a method of treating, ameliorating or preventing a tumor, an immune-related disease, or an infectious disease, comprising administering to a subject in need thereof the fusion protein of the first aspect, the nucleic acid of the second aspect, the expression vector of the third aspect, or the host cell of the fourth aspect.

In a ninth aspect, the present application provides the fusion protein of the first aspect, the nucleic acid of the second aspect, the expression vector of the third aspect, or the host cell of the fourth aspect for use in treating, ameliorating, or preventing a tumor, an immune-related disease, or an infectious disease.

The fusion protein of the CD137 antibody and CD40L of the present application is capable of binding specifically to CD40, has the effects of inducing dendritic cell maturation, activating lymphocytes, etc., and/or the fusion protein of the CD137 antibody and CD40L of the present application can target 4-lBB and activate the signal transduction pathway of 4-lBB to enhance immune responses.

### BRIEF DESCRIPTION OF DRAWINGS

FIG. 1 shows a schematic of the molecular structure of a fusion protein of the CD137 antibody and CD40L of the present application.

FIG. 2 shows the in vitro cell binding activity of a fusion protein of the CD137 antibody and CD40L of the present application, which is determined by flow cytometry. FIG. 2A shows the binding of the fusion protein to the CD40 molecule on the surface of the HEK Blue::CD40L cells; and FIG. 2B shows the binding of the fusion protein to the 4-1BB molecule on the surface of the HEK 293 4-1BB cells.

FIG. 3 shows the in vitro cellular function of the fusion protein of the CD137 antibody and CD40L of the present application, which is determined by a cell function assay. FIG. 3A shows that the fusion protein can bind to the CD40 molecule on the surface of the HEK Blue::CD40L cells and initiate downstream signaling pathways; and FIG. 3B shows that the fusion protein can bind to the 4-1BB molecule on the surface of the HEK 293 4-1BB cells and initiate downstream signaling pathways.

FIG. 4 shows that the fusion protein of the CD137 antibody and CD40L of the present application can promote DC cell maturation, in which FIG. 4A shows that the fusion protein can stimulate the secretion of IL-12 (p40) by DC cells; FIG. 4B shows that the fusion protein can stimulate DC cells to upregulate the expression of MHC class I molecules; FIG. 4C shows that the fusion protein can stimulate DC cells to upregulate the expression of MHC class II molecules; FIG. 4D shows that the fusion protein can stimulate DC cells to upregulate the expression of CD80 molecules; FIG. 4E shows that the fusion protein can stimulate DC cells to upregulate the expression of CD83 molecules; and FIG. 4F shows that the fusion protein can stimulate DC cells to upregulate the expression of CD86 molecules.

FIG. 5 shows the experimental results of in vivo stimulation of the fusion protein of the CD137 antibody and CD40L of the present application in combination with OVA and polyIC:LC in hCD40×h4-1BB KI mice. FIG. 5A shows the experimental results of the in vivo stimulation of the first model protein immune stimulation of the fusion protein on hCD40×h4-1BB KI mice, which is expressed as the percentage of specific CTL (CD3+CD8+CD44+OT1+) in CD8+ cells; FIG. 5B shows the experimental results of the in vivo stimulation of the first model protein immune stimulation of the fusion protein on hCD40×h4-1BB KI mice, which is expressed as the percentage of specific Tem (CD3+CD8+CD44+CD62L−) in CD8+ cells; FIG. 5C shows a summary of the experimental results of the in vivo stimulation of the first model protein immune stimulation of the fusion protein on hCD40×h4-1BB KI mice; FIG. 5D shows the experimental results of the in vivo stimulation of the second model protein immune stimulation of the fusion protein on hCD40×h4-1BB KI mice, which is expressed as the percentage of specific CTL (CD3+CD8+CD44+OT1+) in CD8+ cells; FIG. 5E shows the experimental results of the in vivo stimulation of the second model protein immune stimulation of the fusion protein on hCD40×h4-1BB KI mice, which is expressed as the percentage of specific Tem (CD3+CD8+CD44+CD62L−) in CD8+ cells; and FIG. 5F shows a summary of the experimental results of the in vivo stimulation of the second model protein immune stimulation of the fusion protein on hCD40×h4-1BB KI mice.

FIG. 6 shows the experimental results of the tumor inhibition of the fusion protein of the CD137 antibody and CD40L of the present application in combination with OVA and polyIC:LC against B16-OVA subcutaneous transplanted tumors in hCD40×h4-1BB KI mice. FIG. 6A shows the tumor volume change curves in the B16-OVA tumor model; FIG. 6B shows individual tumor inhibition curves of the fusion protein, showing tumor volume changes in each group, respectively; FIG. 6C shows the tumor weight index of the fusion protein against tumor-bearing (B16-OVA) hCD40×h4-1BB KI mice; FIG. 6D shows the effect of the fusion protein on CTL and Tem in peripheral blood of tumor-bearing (B16-OVA) hCD40×h4-1BB KI mice at the end of the experiment; FIG. 6E shows the effect of the fusion protein on intratumoral CTL and Tem in tumor-bearing (B16-OVA) hCD40×h4-1BB KI mice at the end of the experiment; and FIG. 6F shows the effect of the fusion protein of the CD137 antibody and CD40L on hepatotoxicity in tumor-bearing (B16-OVA) hCD40×h4-1BB KI mice.

FIG. 7 shows the experimental results of the tumor inhibition of the fusion protein of the CD137 antibody and CD40L of the present application in combination with MC38 peptide and polyIC:LC against MC38 subcutaneous transplanted tumors in hCD40×h4-1BB KI mice. FIG. 7A shows the tumor volume change curves in the MC38 tumor model; FIG. 7B shows individual tumor inhibition curves of the fusion protein, showing tumor volume changes in each group, respectively; FIG. 7C shows the tumor weight index of the fusion protein against tumor-bearing (MC38) hCD40×h4-1BB KI mice; FIG. 7D shows the effect of the fusion protein on CTL and Tem in peripheral blood of tumor-bearing (MC38) hCD40×h4-1BB KI mice at the end of the experiment; and FIG. 7E shows the effect of the fusion protein on intratumoral CTL and Tem in tumor-bearing (MC38) hCD40×h4-1BB KI mice at the end of the experiment.

FIG. 8 shows the experimental results of tumor inhibition of MC38 subcutaneous transplanted tumor by intratumoral administration of the fusion protein of the CD137 antibody and CD40L of the present application in hCD40×h4-1BB KI mice. FIG. 8A shows the tumor volume change curves in the MC38 tumor model; FIG. 8B shows individual tumor inhibition curves of the fusion protein, showing tumor volume changes in each group, respectively; FIG. 8C shows the tumor weight index of the fusion protein against tumor-bearing (MC38) hCD40×h4-1BB KI mice; FIG. 8D shows the effect of the fusion protein on Tem and Treg (CD3+CD4+CD25+) in peripheral blood of tumor-bearing (MC38) hCD40×h4-1BB KI mice at the end of the experiment; FIG. 8E shows the effect of the fusion protein on intratumoral Tem and Treg in tumor-bearing (MC38) hCD40×h4-1BB KI mice at the end of the experiment; and FIG. 8F shows the effect of the fusion protein of the CD137 antibody and CD40L on hepatotoxicity in tumor-bearing (MC38) hCD40×h4-1BB KI mice.

FIG. 9 shows the experimental results of tumor inhibition of MC38 subcutaneous transplanted tumor by intratumoral administration of the fusion protein of the CD137 antibody and CD40L of the present application in combination with radiotherapy and polyIC:LC in hCD40×h4-1BB KI mice. FIG. 9A shows the tumor volume change curves in the MC38 tumor model; FIG. 9B shows individual tumor inhibition curves of the fusion protein, showing tumor volume changes in each group, respectively; FIG. 9C shows the tumor weight index of the fusion protein against tumor-bearing (MC38) hCD40×h4-1BB KI mice; FIG. 9D shows the effect of the fusion protein on Tem and Treg (CD3+CD4+CD25+) in peripheral blood of tumor-bearing (MC38) hCD40×h4-1BB KI mice at the end of the experiment; FIG. 9E shows the effect of the fusion protein on intratumoral Tem and Treg in tumor-bearing (MC38) hCD40×h4-1BB KI mice at the end of the experiment; and FIG. 9F shows the effect of the fusion protein of the CD137 antibody and CD40L on hepatotoxicity in tumor-bearing (MC38) hCD40×h4-1BB KI mice.

### DETAILED DESCRIPTION OF INVENTION

The following definitions and methodologies are provided to better define the present application and to guide those of ordinary skill in the art in the practice of the present application. Unless otherwise indicated, the terms used in this application have the meanings commonly understood by those skilled in the art. All patent documents, academic papers, and other publications cited herein are incorporated by reference in their entirety.

### Definition

As used herein, the term “fusion protein” means that two or more genes encoding a functional protein are purposely linked together to express the protein. The fusion protein is a protein product obtained by end-to-end linking the coding regions of two or more genes under artificial conditions and expressing the resultant genes under the control of regulatory sequences.

As used herein, the term “peptide linker” in the context of the application refers to a short peptide used to connect two functional proteins, which may be 3 amino acids (aa) to up to 76 amino acids in length. The peptide linkers may provide some flexibility for each functional protein in the fusion protein to enable them to carry out respective functions thereof.

As used herein, the term “antibody” refers to an immunoglobulin molecule capable of specifically binding to a target via at least one antigen recognition site located in the variable region of the immunoglobulin molecule. The target includes, but is not limited to, carbohydrates, polynucleotides, lipids, and polypeptides. The antibody as used herein includes not only intact (i.e., full-length) antibodies, but also antigen-binding fragments thereof (e.g., Fab, Fab′, F(ab′)₂, and Fv), variants thereof, fusion proteins comprising antibody moieties, humanized antibodies, chimeric antibodies, diabodies, linear antibodies, single-chain antibodies, multispecific antibodies (e.g., bispecific antibodies), and any other modified version of immunoglobulin molecules comprising antigen recognition sites with desired specificity, including glycosylation variants of antibodies, amino acid sequence variants of antibodies, and covalently modified antibodies.

Typically, the whole or full-length antibody comprises two heavy chains and two light chains. Each heavy chain contains a heavy chain variable region (VH) and first, second and third constant regions (CH1, CH2 and CH3). Each light chain contains a light chain variable region (VL) and a constant region (CL). The full-length antibody may be any kind of antibody, such as IgD, IgE, IgG, IgA or IgM (or a subclass thereof), but the antibody need not belong to any particular class. Immunoglobulins can be assigned to different classes depending on the amino acid sequence of the constant domain of the heavy chain of an antibody. Generally, there are five main classes of immunoglobulins: IgA, IgD, IgE, IgG, and IgM, and several of these classes can be further divided into subclasses (isotypes), such as IgG1, IgG2, IgG3, IgG4, IgA1, and IgA2. Heavy chain constant domains corresponding to different classes of immunoglobulins are referred to as a, 6, s, y and p respectively. Subunit structures and three-dimensional structures of different classes of immunoglobulins are well-known.

As used herein, the term “antigen-binding fragment” refers to a portion of the antibody structure that determines the antigen binding capacity. It will be appreciated by those skilled in the art that the major part of the antibody structure that determines the antigen binding capacity is the CDR, which is therefore also the core component of the antigen binding fragment. The antigen binding fragment may comprise a heavy chain variable region (VH), a light chain variable region (VL), or both. Each of VH and VL typically contains three complementarity determining regions, i.e., CDR1, CDR2, and CDR3. Antigen-binding fragments of antibodies can be prepared from intact antibody molecules using any suitable standard technique including, but not limited to, proteolytic digestion or recombinant genetic engineering techniques.

Examples of antigen-binding fragments include, but are not limited to, (1) Fab fragments, which may be monovalent fragments having VL-CL chains and VH-CH1 chains; (2) F(ab′)₂ fragments, which may be divalent fragments having two Fab′ fragments linked by disulfide bridges (i.e., dimers of Fab′) in the hinge region; (3) Fv fragments of VL and VH domains having a single arm of an antibody; (4) a single chain Fv (scFv), which may be a single polypeptide chain consisting of a VH domain and a VL domain via a peptide linker; and (5) (scFv)₂, which may comprise two VH domains linked by peptide linkers and two VL domains that are combined with the two VH domains via disulfide bridges.

As used herein, the term “Fab fragment”, “Fab moiety” or the like refers to an antibody fragment capable of binding to an antigen that is produced after treatment of an intact antibody with papain, including an intact light chain (VL-CL), a heavy chain variable region, and a CH1 fragment (VH-CH1).

It is well-known to those skilled in the art that the complementarity determining regions (CDRs, which are typically CDR1, CDR2 and CDR3) are the regions of the variable regions that have the greatest impact on the affinity and specificity of an antibody. There are two common ways to define the CDR sequences of VH or VL, namely the Kabat definition and the Chothia definition. See, for example, “Sequences of Proteins of Immunological Interest”, National Institutes of Health, Bethesda, MD. (1991); Al-Lazikani et al., J Mol Biol 273:927-948 (1997); and Martin et al., Proc. Natl. Acad. Sci. USA 86:9268-9272 (1989). For the variable region sequences of a given antibody, the CDR region sequences in the VH and VL sequences can be determined according to the Kabat definition or the Chothia definition. In an embodiment of the present application, CDR sequences are defined by Kabat. Herein, CDR1, CDR2 and CDR3 of the heavy chain variable regions are simply referred to as HCDR1, HCDR2 and HCDR3, respectively; and CDR1, CDR2, and CDR3 of the light chain variable regions are simply referred to as LCDR1, LCDR2, and LCDR3, respectively.

For the variable region sequences of a given antibody, the CDR region sequences in the variable region sequences can be analyzed in a number of ways, for example, which can be determined by using the online software Abysis (http://www.abysis.org/).

As used herein, the term “specifically binding” refers to a non-random binding reaction between two molecules, e.g., the binding of an antibody to an epitope, e.g., the ability of an antibody to bind to a specific antigen with an affinity that is at least two times greater than its affinity for a non-specific antigen. However, it should be understood that antibodies are capable of specifically binding to two or more antigens. For example, exemplary antibodies of the present application may specifically bind to CD40 and 4-1BB of human and non-human (e.g., mouse or non-human primates).

As used herein, the term “monoclonal antibody” refers to an antibody obtained from a population of substantially homogeneous antibodies, i.e., the antibodies constituting the population of antibodies are identical to each other, except that less variations that may occur naturally may be present. Monoclonal antibodies are highly specific for a single antigenic epitope. The monoclonal antibodies disclosed herein are not limited to antibody sources or methods of preparation thereof (e.g., by hybridomas, phage selection, recombinant expression, transgenic animals, etc.). The term includes intact immunoglobulins under the definition of “antibody” and fragments thereof, and the like.

As used herein, the term “agonist” refers to a class of molecules such as drugs, enzyme agonists, and proteins that can enhance the activity of another molecule, and promote a certain response. In the context of the present application, when added to a cell, tissue or organism expressing CD40, the fusion protein of the CD137 antibody and CD40L can increase the activity of one or more CD40 by at least about 20%. In some embodiments, molecules having agonist function, such as the fusion protein of the present application, can increase the activity of CD40 by at least 40%, 50%, or 60%, or more. In some embodiments, the release of IL-12 is determined by using a dendritic cell assay to determine the activity of activated antibodies.

As used herein, the term “identity”, in the case of two or more nucleic acids or polypeptides, refers to two or more sequences or subsequences that are the same or have the specified percentage of the same nucleotide or amino acid (i.e., in a specified region, about 60% identity, preferably 65%, 70%, 75%, 80%, 85%, 90%, 91%, 92%, 93%, 94%, 95%, 96%, 97%, 98%, 99%, or more identity when compared and aligned the maximum identity over a comparison window or the specified region), as measured using a BLAST or BLAST 2.0 sequence comparison algorithm with default parameters or by manual alignment and visual inspection (see, e.g., the NCBI website, etc.).

As used herein, the term “CD40 related diseases” includes diseases and/or symptoms associated with the CD40 signaling pathway. Exemplary CD40-related diseases or conditions include, but are not limited to, tumors such as colon tumors, melanoma, leukemia, and lymphoma.

As used herein, the term “K_{D}” refers to the equilibrium dissociation constant of a specific antibody-antigen interaction in the context of the present application.

### DETAILED DESCRIPTION

CD40 signal transduction activates a number of pathways, including NF-κB (nuclear factor-KB), MAPK (mitogen-activated protein kinase) and STAT3 (signal transducer and transcriptional activator-3) (Pype S et al., J Biol Chem. 2000; 275(24):18586-18593) and regulates gene expression by activating activator protein, c-Jun, ATF2 (activating transcription factor-2) and Rel transcription factor (Dadgostar H et al., Proc Natl Acad Sci USA. 2002; 99(3): 1497-1502). Genes activated in response to CD40 signals include various cytokines and chemokines, such as IL-1, IL-6, IL-8, IL-10, IL-12, TNF-α and macrophage inflammatory protein-1α (MIP1α). In certain cell types, the activation of CD40 may result in the production of cytotoxic free radicals (Dadgostar H et al., Proc Natl Acad Sci USA. 2002; 99 (3):1497-1502), COX-2 (cyclooxygenase-2), and NO (nitric oxide).

CD40 is expressed not only on normal immune cells, but also on many malignant cells. For example, CD40 may be overexpressed in B-line NHL, chronic lymphocytic leukemia (CLL), hairy cell leukemia (HCL), Hodgkin's disease (Uckun F M et al., Blood 1990; 76: 2449-2456; O'Grady J T et al., Am J Pathol 1994; 144:21-26), multiple myeloma (Pellat-Deceunynck C et al., Blood 1994; 84(8):2597-2603), bladder cancer, renal cancer, ovarian cancer, cervical cancer, breast cancer, lung cancer, nasopharyngeal cancer, and malignant melanoma (Young L S et al., Immunol Today 1998; 19: 502-506; Ziebold J L et al., Arch Immunol Ther Exp (Warsz) 2000; 48: 225-233; Gladue R et al., J Clin Oncol 2006; 2(18S): 103s) and so on.

In many cases, upon binding to the CD40 molecule on the surface of tumor cells, the antibodies can mediate direct cytotoxicity, causing tumor regression through apoptosis and cell necrosis (Grewal I S et al., Annu Rev Immunol 1998; 16: 111-135; and van Kooten C et al., J Leukoc Biol 2000; 67(1):2-17). In addition to direct tumor suppression, CD40 signaling activation can also rescue the function of antigen-presenting cells in a tumor-bearing host and trigger or restore an immune response to the activation of tumor-associated antigens. It has been reported that CD40 agonists can overcome T cell tolerance in tumor-bearing mice, elicit effective cytotoxic T cell responses against tumor-associated antigens, and enhance the efficacy of anti-tumor vaccines (Eliopoulos A G et al., Mol Cell Biol 2000; 20(15):5503-5515; Tong A W et al., Clin Cancer Res 2001; 7(3):691-703).

CD137 (4-1BB) is also a member of the TNFR family and is a costimulatory molecule on CD8+ and CD4+ T cells, regulatory T cells (Tregs), natural killer T cells (NK (T) cells), B cells, and neutrophils. CD137 is not constitutively expressed on T cells, but is induced to express after activation of the T cell receptors (TCRs), for example, on tumor infiltrating lymphocytes (TILs) (Gros et al., J. Ciin Invest 2014; 124(5):2246-59)). Early signal transduction through CD137 involves K-63 polyubiquitination, which ultimately leads to activation of the nuclear factor (NF)-kB and mitogen activator protein (MAP)-kinase pathways. Signal transduction will increase T cell co-stimulation, proliferation, and cytokine production, mature CD8+ T cell, and prolong CD8+ T cell survival. Agonistic antibodies against CD137 have been shown to promote anti-tumor control of T cells in various preclinical models (Murillo et al., Clin Cancer Res 2008; 14(21):6895-906). Antibodies that stimulate CD137 can induce survival and proliferation of T cells, thereby enhancing anti-tumor immune responses.

The binding of both CD40-expressing APC and CD137-expressing T cells will bring these types of cells into close contact. This in turn can lead to activation of both cell types and effective induction of anti-tumor immunity.

In view of the shortcomings of conventional CD40 antibodies, a novel molecule—the fusion protein of the CD137 antibody and CD40L is prepared by genetic engineering and antibody engineering, and CD137 (4-1BB) is added on the basis of the CD40L activating immune system to further enhance immune responses.

In a first aspect, the present application provides a fusion protein comprising:
a) a Fab fragment specifically binding to 4-lBB molecules;
b) a first CD40L specifically binding to CD40 molecules, the N-terminus of the first CD40L being linked to the C-terminus of the light chain or heavy chain of the Fab fragment by a first peptide linker.

In some embodiments, the fusion protein further comprises:
c) a second CD40L specifically binding to CD40 molecules, the N-terminus of the second CD40L being linked to the C-terminus of the heavy chain or light chain of the Fab fragment by a second peptide linker.

In some embodiments, only one disulfide bond can be formed between the first peptide linker and the second peptide linker, and each of the peptide linkers is independently selected from the group consisting of a peptide linker comprising any of the sequences as set forth in SEQ ID NOs: 24-25 (SEQ ID NO:24: Xaa Pro Pro Cys Pro Ala Pro Glu; SEQ ID NO:25: Glu Pro Ala Pro Cys Pro Pro Xaa, wherein X can be any amino acid other than Cys, or be absent). Any amino acid other than Cys can be a natural amino acid or an unnatural amino acid. Natural amino acids include, but are not limited to, glycine, alanine, valine, leucine, isoleucine, phenylalanine, proline, serine, threonine, histidine, tryptophan, aspartic acid, glutamic acid, lysine, tyrosine, methionine, asparagine, glutamine and arginine.

In some embodiments, the first peptide linker and/or the second peptide linker can be a hinge region of a native antibody, and wherein a mutation that retains only one cysteine can be made to the hinge region.

In some embodiments, the first peptide linker and/or the second peptide linker can be the IgG1 hinge region with C239 being deleted or substituted, or the IgG1 hinge region with C239 being deleted or substituted and the hinge region D234-S252 being inverted. As used herein, the term “substitution” can refer to the substitution of an amino acid at the designated position by any other natural or unnatural amino acid, e.g., the substitution of the cysteine at position 239 by one of the 20 natural amino acids. Natural amino acids include, but are not limited to, glycine, alanine, valine, leucine, isoleucine, phenylalanine, proline, serine, threonine, histidine, tryptophan, aspartic acid, glutamic acid, lysine, tyrosine, methionine, asparagine, glutamine and arginine. Unnatural amino acids include, but are not limited to, D-forms of various natural amino acids, such as D-glycine, D-alanine, and D-valine, as well as derivatives of various natural amino acids, such as hydroxyproline, hydroxylysine, and homoleucine.

In a specific embodiment, the first peptide linker and the second peptide linker are each independently selected from Asp Lys Thr His Thr Xaa Pro Pro Cys Pro Ala Pro Glu Leu Leu Gly Gly Pro Ser or Ser Pro Gly Gly Leu Leu Glu Pro Ala Pro Cys Pro Pro Xaa Thr His Thr Lys Asp, wherein Xaa is absent or represents any amino acid other than Cys, such as one of 20 natural amino acids.

In some embodiments, the first peptide linker and the second peptide linker are the same.

In some embodiments, the first peptide linker and the second peptide linker are different.

In some embodiments, the fusion protein further comprises:
d) a third CD40L specifically binding to CD40 molecules, the N-terminus of the third CD40L being linked to the C-terminus of the first CD40L or the second CD40L by a third peptide linker.

In some embodiments, the third peptide linker comprises one or more of the sequence as set forth in SEQ ID NO: 5, the sequence as set forth in SEQ ID NO:6, a plurality of SEQ ID NOs: 5 linked in series, and a plurality of SEQ ID NOs: 6 linked in series. The plurality includes 2, 3, 4, 5, 6 or more as long as the length thereof is within an acceptable range.

The fusion protein further comprises e) FcBP, wherein the FcBP is linked to the C-terminus of any one or more of the first CD40L, the second CD40L, and the third CD40L. Preferably, the FcBP is linked to the C-terminus of CD40L attached to the light chain to increase the half-life of the fusion protein thus obtained.

In some embodiments, a fourth peptide linker and a fifth peptide linker are also linked between the first peptide linker and the first CD40L and between the second peptide linker and the second CD40L.

In some embodiments, the fourth peptide linker and the fifth peptide linker each independently comprise one or more of the sequence as set forth in SEQ ID NO: 5, the sequence as set forth in SEQ ID NOs:6, a plurality of SEQ ID NOs: 5 linked in series, and a plurality of SEQ ID NO: 6 linked in series. Preferably, the fourth peptide linker and the fifth peptide linker are the same. The plurality includes 2, 3, 4, 5, 6 or more as long as the length thereof is within an acceptable range.

In some embodiments, the 4-1BB molecule and the CD40 molecules are independently derived from a mammal, preferably a non-human primate or human.

In some embodiments, the Fab fragment comprises HCDR1 as set forth in SEQ ID NO: 7, HCDR2 as set forth in SEQ ID NO: 8, and HCDR3 as set forth in SEQ ID NO: 9.

In some embodiments, the Fab fragment comprises the heavy chain variable region as set forth in SEQ ID NO: 13.

In some embodiments, the Fab fragment comprises a light chain variable region having at least about 70%, 75%, 80%, 85%, 86%, 87%, 88%, 89%, 90%, 91%, 92%, 93%, 94%, 95%, 96%, 97%, 98%, 99% or more identity to the amino acid sequence as set forth in SEQ ID NO: 13 and being capable of binding to the 4-1BB molecule.

In some embodiments, the Fab fragment comprises LCDR1 as set forth in SEQ ID NO: 10, LCDR2 as set forth in SEQ ID NO: 11, and LCDR3 as set forth in SEQ ID NO: 12.

In some embodiments, the Fab fragment comprises the light chain variable region as set forth in SEQ ID NO: 14.

In some embodiments, the Fab fragment comprises a light chain variable region having at least about 70%, 75%, 80%, 85%, 86%, 87%, 88%, 89%, 90%, 91%, 92%, 93%, 94%, 95%, 96%, 97%, 98%, 99% or more identity to the amino acid sequence as set forth in SEQ ID NO: 14 and being capable of binding to the 4-1BB molecule.

The amino acid sequence of an antibody is numbered to identify equivalent positions, and there are currently a number of different numbering protocols for the antibody. The Kabat protocol (Kabat et al., 1991) was developed based on the positions of regions of high sequence variation between sequences of the same type of domains. Its numbers differ against the variable domains of the heavy (VH) and light (Vκ and Vκ) chains of an antibody. The Chothia protocol (Al-Lazikani, 1997) is identical to the Kabat protocol, but corrects the positions at which the annotation is inserted around the first VH complementarity determining region (CDR) so that they correspond to the structural ring. The antibodies in the present application are numbered according to the Kabat protocol.

In some embodiments, the first CD40L, the second CD40L, and the third CD40L each independently comprise any of SEQ ID NOs: 1-4.

In some embodiments, the first CD40L, the second CD40L, and the third CD40L each independently comprise a light chain variable region having at least about 70%, 75%, 80%, 85%, 86%, 87%, 88%, 89%, 90%, 91%, 92%, 93%, 94%, 95%, 96%, 97%, 98%, 99%, or more identity to the amino acid sequence as set forth in any of SEQ ID NOs: 1-4 and being capable of binding to the CD40 molecule.

In some embodiments, the fusion protein specifically binds to the CD40 molecule with an affinity of at least 1×10⁻⁸.

In some embodiments, the fusion protein has the function of CD40 agonist and can induce dendritic cell maturation and/or T cell activation.

In some embodiments, the fusion protein disclosed herein can induce dendritic cell maturation. In some embodiments, the fusion protein disclosed herein can induce T lymphocyte activation.

For example, in a specific embodiment, the fusion protein disclosed herein can enhance the ability of dendritic cells to express CD80, CD83, CD86, MHCI, and/or MHC II molecules. In a specific embodiment, the fusion protein disclosed herein can stimulate the secretion of cytokines by dendritic cells and adherent monocytes, including, but not limited to, IL-8, IL-12, IL-15, IL-18, and IL-23.

In a specific embodiment, the fusion protein disclosed herein can activate antigen-specific T cells, e.g., activate specific T cells in hCD40×h4-1BB KI mice.

In some embodiments, the fusion protein disclosed herein can inhibit tumor growth. For example, in a specific embodiment, the fusion protein disclosed herein can inhibit the growth of melanoma. In a specific embodiment, the above fusion protein inhibits tumor growth by at least 50%. In some embodiments, inhibition of tumor growth can be detected 7 days after a tumor-bearing individual is treated with the fusion protein disclosed herein. In other embodiments, inhibition of tumor growth can be detected 4 days after initial antibody treatment.

In some embodiments, the fusion protein specifically binds to the 4-1BB molecule with an affinity of at least 1×10⁻⁸.

In some embodiments, the fusion protein has the function of 4-1BB agonist and can induce T cell activation.

The binding force between molecules is referred to herein as affinity, which is essentially a non-covalent force. It reflects the binding ability between molecules (for example, between antibodies and antigens, and between receptors and ligands). Methods for determining the affinity between molecules are well-known in the art and include, but are not limited to, biolayer interferometry (BLI), solid phase radioimmunoassay (SP-RIA), equilibrium dialysis, binding antigen precipitation, radioimmunoassay (RIA), enzyme-linked immunosorbent assay (ELISA), and surface plasmon resonance (SPR). The magnitude of the affinity can be expressed as an affinity constant K_{D}. The higher the affinity constant K_{D} is, the stronger the binding ability of the two is.

In the fusion protein of the present application, the Fab fragment capable of specifically binding to the 4-1BB molecule can be linked to CD40L, preferably the extracellular region of CD40L, such as the extracellular region of CD40L as set forth in any of SEQ ID NOs: 1-4 at the C-terminus of the heavy chain of the Fab fragment by a peptide linker, and the peptide linker is preferably the IgG1 hinge region with the C239 deletion mutation. Specifically, SEQ ID NO: 1 is the extracellular region CD40L 113-261 of CD40L; SEQ ID NO: 2 is the extracellular region CD40L 116-261 of CD40L; SEQ ID NO: 3 is the extracellular region CD40L 119-261 of CD40L; and SEQ ID NO: 4 is the extracellular region CD40L 121-261 of CD40L. In some embodiments, the Fab fragment capable of specifically binding to the 4-1BB molecule can also be linked to CD40L, preferably the extracellular region of CD40L, such as the extracellular region of CD40L as set forth in any of SEQ ID NOs: 1-4 at the C-terminus of the light chain of the Fab fragment by a peptide linker, and the peptide linker is preferably the IgG1 hinge region with the C239 deletion mutation. Therefore, the fusion protein construct of the present application can comprise two copies of the extracellular region of CD40L. In some specific embodiments, the fusion protein comprising two copies of the extracellular region of CD40L can also comprise another copy of the extracellular region of CD40L, which can be linked by another linker to the C-terminus of either of the existing two extracellular regions of CD40L to form a fusion protein comprising three copies of the extracellular region of CD40L.

In a second aspect, the present application provides a nucleic acid encoding the fusion protein of the first aspect.

In a preferred embodiment, the nucleic acid may be a codon optimized nucleic acid suitable for expression in host cells. For example, according to the degeneracy of the codon, it still encodes the same protein. Methods for codon optimization according to the host cells used are well-known to those skilled in the art.

In a third aspect, the present application provides an expression vector comprising the nucleic acid of the second aspect.

Any suitable expression vectors can be used. For example, prokaryotic cloning vectors include plasmids from E. coli, such as colEl, pCR1, pBR322, pMB9, pUC, pKSM, and RP4. Prokaryotic vectors also include phage DNA such as M13 and derivatives of other filamentous single-stranded DNA phages. An example of a vector useful for yeast is the 2 plasmid. Suitable vectors for expression in mammalian cells include the following well-known derivatives: SV-40, adenovirus, retrovirus-derived DNA sequences, and shuttle vectors derived from combinations of functional mammalian vectors, such as those described above, and functional plasmid and phage DNA.

Additional eukaryotic expression vectors are known in the art (e.g., P J. Southern & P. Berg, J. Mol. Appl. Genet, 1:327-341 (1982); Subramani et al., Mol. Cell. Biol, 1: 854-864 (1981); Kaufinann & Sharp, “Amplification And Expression of Sequences Cotransfected with a Modular Dihydrofolate Reductase Complementary DNA Gene,” J. Mol. Biol, 159:601-621 (1982); Kaufhiann & Sharp, Mol. Cell. Biol, 159:601-664 (1982); Scahill et al., “Expression And Characterization Of The Product Of A Human Immune Interferon DNA Gene In Chinese Hamster Ovary Cells,” Proc. Nat'l Acad. Sci USA, 80:4654-4659 (1983); Urlaub & Chasin, Proc. Nat'l Acad. Sci USA, 77:4216-4220, (1980), which is incorporated herein by reference in its entirety).

Expression vectors useful in the application comprise at least one expression control sequence operably linked to a DNA sequence or fragment to be expressed. The control sequence is inserted into a vector to control and regulate the expression of cloned DNA sequences. Examples of useful expression control sequences are the lac system, the trp system, the tac system, the trc system, the major operon and promoter region of the phage Lamda, the control region of the fd coat protein, the glycolytic promoter of the yeast, such as the promoter of 3-phosphoglycerate kinase, the promoter of the yeast acid phosphatase, such as Pho5, the promoter of the yeast alpha mating factor, and promoters derived from a polyomavirus, an adenovirus, a retrovirus, and a simian virus, such as the early and late promoters of SV40, and other sequences known to control gene expression of prokaryotic or eukaryotic cells and viruses or combinations thereof.

In a fourth aspect, the present application provides a host cell comprising the nucleic acid of the second aspect or the expression vector of the third aspect.

In some embodiments, the host cell is a mammalian cell. The mammalian cell may include, but are not limited to, a CHO cell, a NS0 cell, a SP2/0 cell, a HEK293 cell, a COS cell, and a PER.C6 cell. One skilled in the art will be able to select suitable host cells as desired.

In a fifth aspect, the present application provides a method of preparing the fusion protein of the first aspect, comprising:
a) culturing the host cell of the fourth aspect; and
b) recovering the fusion protein from the host cells or from the culture supernatant of the host cells.

In a sixth aspect, the present application provides a pharmaceutical composition comprising the fusion protein of the first, the nucleic acid of the second aspect, the expression vector of the third aspect, or the host cell of the fourth aspect, and a pharmaceutically acceptable carrier.

The pharmaceutical composition of the sixth aspect may be prepared in a desired dosage form according to conventional methods in the pharmaceutical field. In some embodiments, the pharmaceutical composition is preferably a liquid or suspension dosage form.

In some embodiments, the pharmaceutically acceptable carrier is a carrier that does not impair the viability and function of an immune cell, and does not affect specific binding of an antibody or antigen-binding fragment thereof to an antigen, including, but not limited to, cell culture media, buffers, physiological saline, balanced salt solutions, and the like. Examples of buffers include isotonic phosphates, acetates, citrates, borates, carbonates, and the like. In a specific embodiment, the pharmaceutically acceptable carrier is phosphate buffer containing 1% serum.

The fusion protein and pharmaceutical composition disclosed herein can be used to treat, ameliorate, or prevent a tumor, an immune-related disease, or an infectious disease in a subject.

The fusion protein and pharmaceutical compositions disclosed herein can be administered in any suitable manners. Preferably, the fusion protein and pharmaceutical composition of the present application are administered by injection (e.g., subcutaneous, intravenous, intratumoral, intraarterial, intramuscular, intradermal, intraperitoneal, or intrathecal). Preferably, the fusion protein and pharmaceutical composition of the present application are administered intravenously. For the fusion protein and pharmaceutical composition of the present application, suitable pharmaceutically acceptable carriers for injection can include any isotonic carrier such as physiological saline (water containing about 0.90% w/v NaCl, water containing about 300 mOsm/L NaCl, or about 9.0 g NaCl per liter of water), NORMOSOL R electrolyte solution (Abbott, Chicago, IL), PLASMA-LYTE A (Baxter, Deerfield, IL), water containing about 5% glucose, or lactated Ringer's solution. In a specific embodiment, the pharmaceutically acceptable carrier is replaced with human serum albumin.

The pharmaceutical composition of the sixth aspect may further comprise a second agent for treating, ameliorating, or preventing a tumor, an immune-related disease, or an infectious disease in a subject.

In a seventh aspect, the present application provides use of the fusion protein of the first aspect, the nucleic acid of the second aspect, the expression vector of the third aspect, or the host cell of the fourth aspect in the manufacture of a medicament for treating, ameliorating, or preventing a tumor, an immune-related disease, or an infectious disease.

In an eighth aspect, the present application provides a method for treating, ameliorating, or preventing a tumor, an immune-related disease, or an infectious disease, comprising administering to a subject in need of the fusion protein of the first aspect, the nucleic acid of the second aspect, the expression vector of the third aspect, or the host cell of the fourth aspect.

In some embodiments, the method further comprises administering a second agent for treating, ameliorating, or preventing a tumor, an autoimmune disease, or an infectious disease.

For the treatment, amelioration or prevention of a tumor, the fusion protein of the first aspect, the nucleic acid of the second aspect, the expression vector of the third aspect or the host cell of the fourth aspect can also be administered to a subject before, simultaneously with or after radiotherapy and/or chemotherapy is performed on the subject.

In a ninth aspect, the present application provides the fusion protein of the first aspect, the nucleic acid of the second aspect, the expression vector of the third aspect, or the host cell of the fourth aspect for use in treating, ameliorating, or preventing a tumor, an immune-related disease, or an infectious disease.

“Treatment” refers to both therapeutic treatment and prophylactic or preventive measures aimed at preventing or slowing (alleviating) the target pathology state or condition. Subjects in need of treatment include those in which the condition already exists, as well as those in which the condition will develop or is intended to be prevented. Thus, a subject to be treated herein has been diagnosed with or tend to have or predisposed to the condition.

As used herein, the term “subject” refers to a mammal, including but not limited to primates, cattle, horse, pig, sheep, goat, dog, cat, and rodent such as rat and mouse. Preferably, the mammal is a non-human primate or human. A particularly preferred mammal is human.

In certain embodiments, the tumor is primary cancer or metastatic cancer. In a specific embodiment, the tumor is selected from the group consisting of lung cancer such as non-small cell lung cancer, colorectal cancer, pancreatic cancer, mesothelioma, bladder cancer, hematopoietic cancer such as leukemia, breast cancer, gastric cancer, adenocarcinoma of the gastro-oesophageal junction, non-Hodgkin's lymphoma, Hodgkin's lymphoma, anaplastic large cell lymphoma, head and neck cancer such as head and neck squamous cell carcinoma, malignant glioma, renal cancer, melanoma, prostate cancer, bone cancer, bone giant cell tumor, pancreatic cancer, sarcoma, liver cancer, skin squamous cell carcinoma, thyroid cancer, cervical cancer, nasal pharynx cancer, endometrial cancer, or metastatic cancer of the above-mentioned tumor.

In certain embodiments, the immune-related disease may include systemic lupus erythematosus, rheumatoid arthritis, scleroderma, systemic vasculitis, dermatomyositis, autoimmune hemolytic anemia, and the like.

In certain embodiments, the infectious disease includes respiratory tract contagion disease, gastrointestinal tract contagion disease, blood contagion disease, body surface contagion disease, sex contagion disease, and the like. In a specific embodiment, the infectious disease may include, but is not limited to, influenza, tuberculosis, HPV infection, colitis, mumps, measles, pertussis, ascarid, bacterial dysentery, hepatitis A, hepatitis B, malaria, epidemic encephalitis B, filariasis, schistosomiasis, trachoma, rabies, tetanus, gonorrhea, syphilis, AIDS, and the like.

As used herein, a “therapeutically effective amount” can be determined as desired, and one of ordinary skill in the art can readily grasp the amount actually required, for example, depending on the weight, age, and condition of the patient.

In this specification and the claims, the terms “comprising,” and “comprises” and “comprise” mean “including, but not limited to” and are not intended to exclude other parts, additions, components or steps.

It is to be understood that the features, characteristics, components, or steps described in a particular aspect, embodiment, or example of the application are applicable to any other aspects, embodiments, or examples described herein, unless indicated otherwise.

The above disclosure generally describes the present application, and the examples are further illustrative of the application and should not be construed as limiting the application. A detailed description of conventional methods, such as those used to construct vectors and plasmids, methods of inserting genes encoding proteins into vectors and plasmids, or methods of introducing plasmids into host cells, are excluded from the examples. Such methods are well-known to those of ordinary skill in the art and are described in a number of publications. See, for example, Sambrook, J., Fritsch, E F. and Maniais, T. (1989) Molecular Cloning: A Laboratory Manual, 2nd edition, Cold spring Harbor Laboratory Press.

### EXAMPLES

The following examples are used to illustrate the present application, but are not intended to limit the scope of the present application. Modifications or substitutions of the methods, steps or conditions of the present application are intended to fall within the scope of the present application without departing from the spirit and essence thereof.

Unless otherwise specified, the chemical reagents used in the examples are all conventional commercially available reagents, and the technical means used in the examples are conventional means well-known to those skilled in the art.

### Example 1: Preparation, Expression and Identification of Fusion Protein of CD137 Antibody and CD40L

### Preparation of Fusion Protein Vector

The anti-CD137 VH-CH1 fragment (referring to Patent No. US-2019-0284292-A1 for the sequence), the anti-CD137 VL-Cκ fragment (referring to Patent No. US-2019-0284292-A1 for the sequence), the CD40L fragment (including CD40L P1 (G4S-CD40L), CD40L P2 (G4S-G3S-CD40L), and CD40L P3 (G4S-CD40L) fragments, the extracellular region CD40L 116-231 being used for CD40L), and the LFcBP fragment were obtained by PCR amplification. The CD137 VH-CH1, CD40L P1, and CD40L P2 fragments were inserted into the fully synthetic expression vector pQKX1 (General Biological Systems (Anhui) Co., Ltd.), and the resulting product was designated as pQK VH; and the CD137 VL-Cκ, CD40L P3, LFcBP fragments were inserted into the fully synthetic expression vector pQKX2, and the resulting product was designated as pQK VL.

### 1.1 Preparation of Expression Vector of Fusion Protein of CD137 Antibody and CD40L

### 1.1.1 Construction of pQK VH and pQK VL of Fusion Protein of CD137 Antibody and CD40L

CD137 VH-CH1 and CD137 VL-Cκ were amplified from pQK Triad2 H and pQK Triad2 L as templates using the Gold Mix PCR kit (TSINGKE Corporation) according to the instructions of manufacturer, and the amplified products thereof were about 5.5 kb and 6.8 kb in size, respectively. The CD40L P1, CD40L P2 and CD40L P3 were amplified from the vector HG10239-CH as a template using the Gold Mix PCR kit (TSINGKE Corporation) according to the instructions of manufacturer, and the amplified products were about 500 bp in size. The LFcBP was amplified from the vector pUC57 LFcBP as a template using the Gold Mix PCR kit (TSINGKE Corporation) according to the instructions of manufacturer, and the amplified product was about 90 bp in size. The fully synthetic vector pQKX1 (General Biological Systems (Anhui) Co., Ltd.) was digested with the restriction enzymes EcoRI (NEB, R3101S) and BspQI (NEB, R0712L), and the three resulting PCR amplification products (ligation sequence from 5′ to 3′: CD137 VH-CH1-CD40L P1-CD40L P2) and the digested vector were recombinantly ligated with the BM seamless clone kit (Boeder Corporation) according to the instructions of manufacturer to obtain the expression vector pQK VH for the heavy chain. Meanwhile, the fully synthetic vector pQKX2 (General Biological Systems (Anhui) Co., Ltd.) was digested with the restriction enzymes EcoRI (NEB, R3101S) and SapI (NEB, R0712S), and the three resulting PCR amplification products (ligation sequence from 5′ to 3′: CD137 Antibody VL-x-CD40L P3-LFcBP) and the digested vector were recombinantly ligated with the BM seamless clone kit (Boeder Corporation) according to the instructions of manufacturer to obtain the expression vector pQK VL for the light chain.

The primer pairs for PCR amplification were as follows:

Primers for amplifying pQK VH fragment

| | |
|---|---|
| 106VH-G4S | 5′-TGTGGCTGAGAGGTGCCAGATGTGAAGTGCAGCTGGTTCAGTCTGGCGCCGAA-3′ |
| F | (SEQ ID NO: 16) |
| 106VH/VL | 5′-CGCAATTTGAGGATTCTGATCACCGCTGCCGCCGCCGCCTGACGGTCC-3′ (SEQ ID |
| R | NO: 17) |

Primers for amplifying CD40L P1/CD40L P2/CD40L P3 fragment

| | |
|---|---|
| CD40L | 5′-GACCGTCAGGCGGCGGCGGCAGCGGTGATCAGAATCCTCAAATTGC-3′ (SEQ ID |
| F | NO: 18) |
| CD40L G4S | 5′-CAATTTGAGGATTCTGATCACCAGAGCCTCCACCAGAGCCTCCACCCCCGAGTTT-3′ |
| R | (SEQ ID NO: 19) |
| CD40L GS | 5′-CAAACTCGGGGGTGGAGGCTCTGGTGGAGGCTCTGGTGATCAGAATCCTCAAAT-3′ |
| F | (SEQ ID NO: 20) |
| CD40L | 5′-TGATTATGATCAATGAATTCCTAGAGTTTGAGTAAGCCAAAGGACGTG-3′ (SEQ ID |
| R | NO: 21) |

Primers for amplifying LFcBP fragment

| | |
|---|---|
| LFcBP | 5′-ACGTCCTTTGGCTTACTCAAACTCCAGAGATTTGTGACCGGCCACTT-3′ (SEQ ID |
| F | NO: 22) |
| LFcBP | 5′-CTGATTATGATCAATGAATTCTCATCCATTAGCAGGATACAGGC-3′ (SEQ ID NO: 23) |
| R | |

### 1.1.2 Amplification and Preparation of Recombinant Plasmid

The expression vector pQK VH for the heavy chain and the expression vector pQK VL for the light chain obtained as above were transformed into DH5a competent cells, respectively. After the clones were picked and identified, they were cultured in LB medium containing ampicillin (final concentration of 100 mg/L) for 16 hours at 37° C. with shaking under 200 rpm. Bacteria were collected by centrifugation at 8000×g for 20 minutes. The plasmid was isolated and extracted using NucleoBond Xtra Midi kit (Macherey-nagel) according to the instructions of manufacturer, and eluted with 1 mL of sterile ultrapure water.

Finally, the plasmid concentration was determined using a Nanodrop microspectrophotometer.

### 1.2 Expression of Antibody

### 1.2.1 Transfection of Fusion Protein

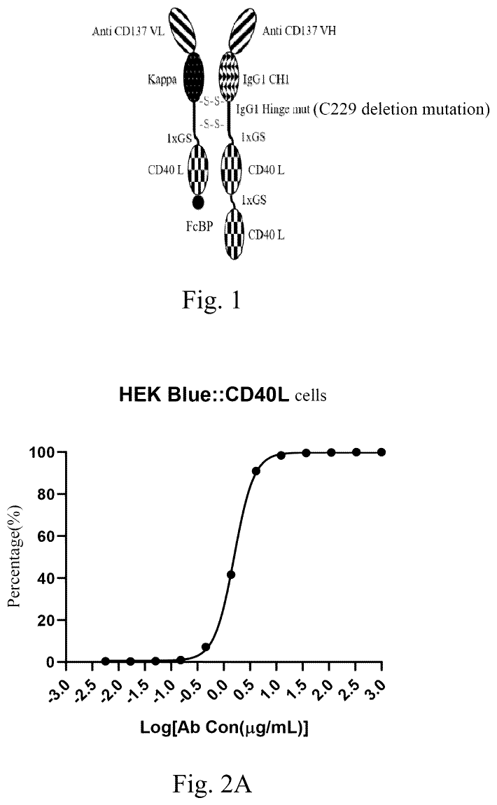

20 μL HEK 293Fv cells (Shanghai Opmel Biotechnology Co., Ltd.) were sampled and counted. The cells were diluted to 1.5×10⁶ cells/mL in preheated OPM-293 CD05 medium (Shanghai Opmel Biotechnology Co., Ltd., 81075-001), and cultured on a shaker at 37° C. for 24 hours. The plasmid was diluted with a 10% transfection volume of fresh OPM-293 CD05 medium and the final plasmid concentration was 1 μg/mL on the basis of the transfected cell volume. 3 mg/mL of PEI (Sigma, 765090) was added to the diluted plasmid at 1/1000 of the cell volume, vortexed immediately for 10 seconds and placed at the room temperature for 15 minutes. The plasmid/PEI mixture was added dropwise to the cell culture medium and the culture flask was gently shaken while adding dropwise. The culture flask was placed on a shaker for culture, 5% CDF08 medium (Opmel, F81288-001)+1% Gluc (Sigma, G8769) was supplemented on day 1, 1% Gluc was supplemented on day 3, and the sample was collected on days 7 to 10. This protein was the fusion protein of the CD137 antibody and CD40L expressed by plasmids pQK VH and pQK VL. The antibody fusion protein was designated as IMB071703 and the structure thereof was shown in FIG. 1.

### 1.2.2 Expression and Purification of Fusion Protein

After 7-10 days of culture, the cell supernatant was taken and centrifuged at 3500 rpm for 30 min. The supernatant was collected. The antibody was purified using the Capto L affinity column (Cytiva, 17547803). The fusion protein was captured from the culture supernatant by the AKTA PURE affinity purification system (GE) using the 5 ml Capto L purification column. The flow rate was set at 3 mL/min, and the purification column was equilibrated with a 5CV 20 mM PB+150 mM NaCl, pH 7.4 equilibrium liquid. After the column was equilibrated, the sample was loaded. When the loading was completed, 20 mM PB+150 mM NaCl, pH 7.4 was used for rinse. Once the rinse was completed, 50 mM citric acid, pH 3.0 eluent was used for elution. The fusion protein was collected and neutralized with 1M Tris-HCl, pH9.0.

### 1.3 Affinity of Fusion Protein Determined by Fortebio

The affinity constant K_{D} of the purified fusion protein was determined using molecular interaction instrument Fortebio Octet QK (Molecular Devices company). After the Ni-NTA sensor (PALL, 18-5102) was coated with 4-1BB protein (BioImmunoah, FZ00401) and CD40 protein (SinoBiological, 10744-H08H), the binding and dissociation curves of the fusion protein were detected and fitted to obtain the binding and dissociation constants. The binding and dissociation constants of the test antibodies were shown in Table 4.

TABLE 4
Assay results of affinity constant of fusion protein

| Antigen | Kₒₙ (1/Ms) | K_{off} (1/s) | K_{D} (M) | R² |
|---|---|---|---|---|
| CD40 | 1.96E+04 | 6.54E−04 | 3.33E−08 | 0.997686 |
| 4-1BB | 1.10E+05 | 1.31E−03 | 1.19E−08 | 0.97082 |

Note:
Dissociation curve was absent for the reference antibody and the data were for reference only.

### Example 2: In Vitro Cell Binding Assay of Fusion Protein of CD137 Antibody and CD40L

The flow cytometry assay was used to determine the binding activity of the fusion protein of the present application to cell lines stably expressing human CD40 (HEK Blue::CD40L cells, InvivoGen, hkb-cd40) at different concentrations.

One bottle (T75) of HEK Blue::CD40L cells (70-80% of cell confluence) was collected, and the supernatant was discarded. The cells were washed once with 5 mL PBS (Servicebio, G4202-500 mL), and digested with 1 mL 0.25% of trypsin (Gibco, 25200-072) for 30s. 5 mL of DMEM (Corning, 10-013-CV) containing 10% FBS was added to blow evenly, and the mixture was centrifuged at 1000 rpm/min for 5 min, the supernatant was discarded, and 2% FBS (Gemini, 900-108) in PBS (FACS buffer) was added to resuspend cells for cell counting. The HEK Blue::CD40L cells were diluted to 1×10⁶ cells/mL, and added to the flow tubes at 50 μL/tube. The HEK Blue::CD40L cells were grouped into blank, secondary antibody groups and fusion protein gradient dilution groups (starting at 1000 μg/mL with three-fold gradient dilution of 11 points). The fusion protein to be detected was respectively added to the flow tubes of each group at 50 μL/tube and incubated at 4° C. for 30 min in the dark. 3 mL FACS buffer was used to wash the mixture twice and the supernatant was discarded. APC anti-human IgGκ (Biolegend, 392708) in 10 μL/test (v/v=1/20 dilution) was added separately depending on groups and the mixture was incubated at 4° C. for 30 min in the dark. 3 mL FACS buffer was used to wash the mixture once and the supernatant was discarded. The cells were suspended with 300 μL/tube of 2% FBS in PBS (FACS buffer), and detected by flow cytometry.

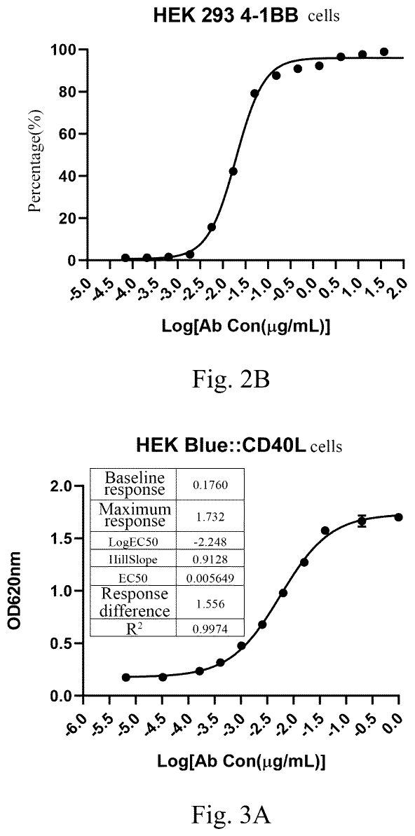

The results showed that the fusion protein was bound to the extracellular region of the human CD40 antigen, which was positively correlated with the fusion protein concentration. The results of this experiment were shown in FIG. 2A.

The FACS assay was used to determine the binding activity of the fusion protein of the present application to cell lines stably expressing human 4-1BB (HEK 293 4-1BB cells, Jiman Biotechnology, CM-C04832) at different concentrations.

One bottle (T75) of HEK 293 4-1BB cells (70-80% of cell confluence) was collected, and the supernatant was discarded. The cells were washed once with 5 mL PBS, and digested with 1 mL 0.25% of trypsin for 30s. 5 mL of DMEM containing 10% FBS was added to blow evenly, and the mixture was centrifuged at 1000 rpm/min for 5 min, the supernatant was discarded, and 2% FBS in PBS (FACS buffer) was added to resuspend cells for cell counting. The HEK 293::4-1BB cells were diluted to 1×10⁶ cells/mL, and added to the flow tubes at 50 μL/tube. The HEK 293::4-1BB cells were grouped into blank, secondary antibody groups and fusion protein gradient dilution groups (starting at 1000 μg/mL with three-fold gradient dilution of 13 points). The fusion protein to be detected was respectively added to the flow tubes of each group at 50 μL/tube and incubated at 4° C. for 30 min in the dark. 3 mL FACS buffer was used to wash the mixture twice and the supernatant was discarded. APC anti-human IgGκ in 10 μL/test (v/v=1/20 dilution) was added separately depending on groups and the mixture was incubated at 4° C. for 30 min in the dark. 3 mL FACS buffer was used to wash the mixture once and the supernatant was discarded. The cells were suspended with 300 μL/tube of 2% FBS in PBS (FACS buffer), and detected by flow cytometry.

The results showed that the fusion protein was bound to the extracellular region of the human 4-1BB antigen, which was positively correlated with the fusion protein concentration. The results of this experiment were shown in FIG. 2B.

### Example 3: In Vitro Functional Assay of Fusion Protein of CD137 Antibody and CD40L

The NF-κB reporter system of HEK Blue::CD40L cells was used for screening. The fusion protein bound to CD40 on the surface of HEK Blue::CD40L cells, thereby activating the downstream signalling pathway NF-κB and resulting in the secretion of alkaline phosphatase (SEAP). The alkaline phosphatase was reacted with Qunati-Blue (InvivoGen, rep-qbs) and the OD values were detected by a multifunctional reader (BMG LABTECH GmbH, CLARIOstar) at the wavelength of 620 nm, so as to detect the in vitro functional activity of the fusion protein.

One bottle (T75) of HEK Blue::CD40L cells (70-80% of cell confluence) was collected, and the supernatant was discarded. The cells were washed once with 5 mL PBS, and digested with 1 mL 0.25% of trypsin for 30s. 3 mL of DMEM (inactivated serum, inactivating in 56° C. water bath for 30 min) containing 10% FBS was added to blow evenly, and the mixture was centrifuged at 1000 rpm/min for 5 min, the supernatant was discarded, and 10% FBS (inactivated serum, inactivating in 56° C. water bath for 30 min) in DMEM was added to resuspend cells for cell counting. The HEK Blue::CD40L cells were diluted to 2×10⁵ cells/mL, and added to a 96-well clear plate (Thermo, 167008) at 100 μL/well. The plate was incubated in a cell incubator (Thermo) at 37° C. and 5% CO₂ for 30 min. The diluted fusion proteins were added to the plate at 100 μL/well. The 96-well plate was incubated in the cell incubator at 37° C. and 5% CO₂ for 20h. 160 μL Quanti-Blue (thawing in advance) was added to the the 96-well plate and 40 μL cell supernatant was taken and added to the 96-well plate. The plate was incubated in the cell culture incubator at 37° C. for 1h, and the OD values were detected by the multifunctional reader at the wavelength of 620 nm.

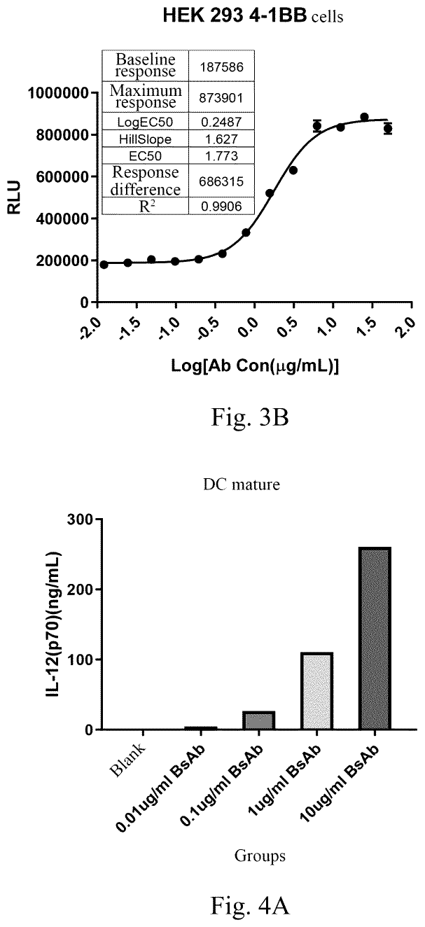

The results showed that the fusion protein can bind to the extracellular region of the human CD40 antigen and stimulate the downstream signalling pathway with an EC50 value of 0.005649 μg/ml, R2=0.9974. The results of this experiment were shown in FIG. 3A.

The NF-κB reporter system of HEK 293 4-1BB cells was used for screening. The fusion protein bound to 4-1BB on the surface of HEK 293 4-1BB cells, thereby activating the downstream signalling pathway NF-κB and resulting in the activation of the luciferase signalling pathway. ONE-Glo™ Luciferase Assay System (Promega, E6120) was reacted with luciferase and the corresponding signal values of the whole spectrum were detected by a multifunctional reader, so as to detect the in vitro functional activity of the antibody.

One bottle (T75) of HEK 293 4-1BB cells (70-80% of cell confluence) was collected, and the supernatant was discarded. The cells were washed once with 5 mL PBS, and digested with 1 mL 0.25% of trypsin for 30s. 4 mL of DMEM containing 10% FBS was added to blow evenly. The cells were collected and the mixture was centrifuged at 1000 rpm/min for 5 min, the supernatant was discarded, and 1% FBS (inactivated serum, inactivating in 56° C. water bath for 30 min) in DMEM was added to resuspend cells for cell counting. The HEK 293 4-1BB cells were diluted to 4×10⁵ cells/mL, and added to a 96-well plate with black wall and transparent bottom (PerkinElmer™, 6005182) at 50 μL/well. The diluted proteins were added in a system of 50 μL/well (the culture system was added to 100 μL/well according to groups). The 96-well plate was incubated in a cell incubator at 37° C. and 5% CO₂ for 5h. ONE-Glo (equilibrated at room temperature for 5 min after thawing) was added to the 96-well plate with black wall and transparent bottom (taken out in advance and equilibrated at room temperature for 5-10 min) at 100 μL/well, and was incubated at room temperature for 5 min in the dark. The response signal values were detected by a multifunctional reader (using ADCC program).

The results showed that the fusion protein can bind to the extracellular region of the human 4-1BB antigen and stimulate the downstream signalling pathway with an EC50 value of 1.773 μg/ml, R2=0.9906. The results of this experiment were shown in FIG. 3B.

### Example 4: Promotion of Dendritic Cell Maturation by Fusion Protein of CD137 Antibody and CD40L

Whether the fusion protein of the present application can promote dendritic cell maturation and regulate the expression of MHC class I molecules, MHC II, CD80, CD83 and CD86 and secretory factors on the surface of dendritic cells is determined by the maturation experiment of dendritic cells.

Peripheral blood was collected from healthy volunteers using EDTA·K2anticoagulant blood collection tubes (2 mL, GE, 367863). The Buffy coat was isolated by Ficoll (GE, 17-1440-02) density gradient centrifugation. Specifically, the blood was diluted with sterile PBS at a ratio of 1:2 (v/v) of anti-coagulant and PBS. The diluted blood was slowly added to the Ficoll liquid level at a ratio of 20:15 (v/v) of the diluted blood and Ficoll. and centrifugated at 800 g for 20 min (rise 3 down 1) at room temperature. The buffy coat was aspirated and washed twice with 1×PBS. The supernatant was discarded. The buffy coat was washed twice with RPMI 1640 containing 10% FBS, the volume of which is twice that of the buffy coat, and centrifuged at 400 g for 5 min. The supernatant was discarded. The cells were resuspended with 5 mL RPMI 1640 containing 10% FBS and counted.

PBMCs were diluted to 2×10⁶ cells/mL with RPMI 1640 containing 10% FBS and added to a 6-well plate at 4 mL/well and incubated for 1-2 h at 37° C. The unadhered cells were gently wash off with 1640 containing 10% FBS at 5 mL/well. Serum-free DC medium was used to prepare cytokine induction medium: 1000 U/mL of GM-CSF (Pepro Tech, 300-03)+500 U/mL of IL-4 (Pepro Tech, 200-04). The cells were incubated at 37° C. for 6 days and the medium was changed every 2-3 days.

ImDC cells were collected, washed once with RPMI 1640 containing 10% FBS, and resuspended for counting. Cells were diluted to 1×10⁶ cells/mL, and plated into a U-shaped bottom 96-well plate (Thermo, 168136) at 100 μL/well. RPMI 1640 containing 10% FBS, 1 μg/mL LPS (Sigma-Aldrich, L4391) and different concentrations of fusion proteins were respectively added to the 96-well plate at 100 μL/well, and were incubated at 37° C. for 48-72 h.

The 96-well plate was centrifuged at 1000 rpm/min for 5 min at 4° C. The cell supernatant was taken and IL-12 (p40) was detected using a Human IL-12 p40 ELISA Kit (MultiSciences Biotech Co., Ltd., EK1183-96). Cells were collected depending on groups, washed twice with 3 mL FACS buffer, added with anti-human CD11c (Biolegend, 301626), anti-human MHC class I (HLA-ABC) molecule (Biolegend, 311426), anti-human MHC class II (HLA-DR) molecule (Biolegend, 307606), anti-human CD80 (Biolegend, 305208), anti-human CD83 (Biolegend, 305325) and anti-human CD86 (Biolegend, 305412), and incubated at 4° C. for 30 min, washed once with 2% FBS in 1×PBS, and resuspended with 2% FBS in 1×PBS at 300 μL/tube·MHC class I molecules, MHC class II molecules, CD80, CD83 and CD86 were detected by flow cytometry.

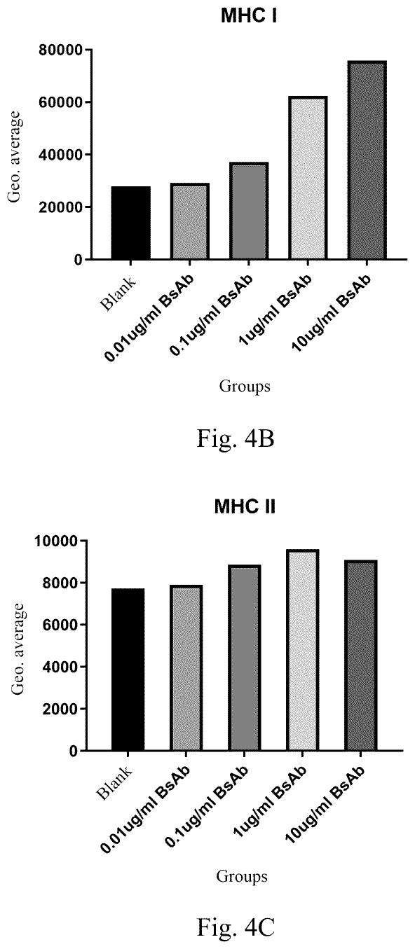

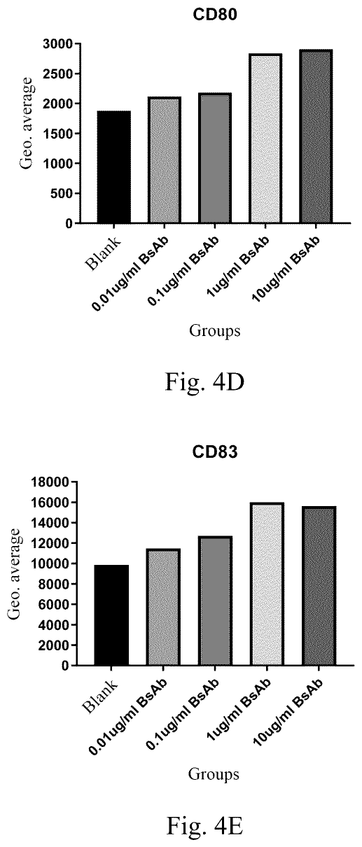

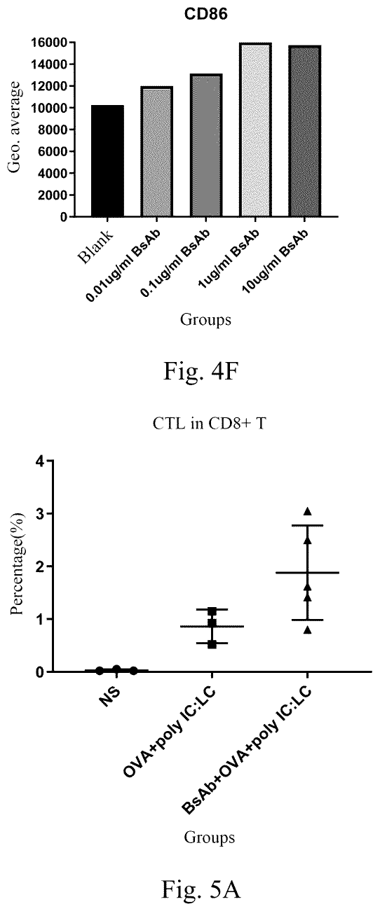

The results were shown in FIG. 4. The fusion protein can promote dendritic cell maturation, stimulate the secretion of IL-12 (P40) by dendritic cells (FIG. 4A), and up-regulate the expression of MHC class I molecules, MHC class II molecules, CD80, CD83, and CD86 on the surface of dendritic cells (FIGS. 4B-F).

### Example 5. In Vivo Stimulation Assay of Fusion Protein in Mice with Model Antigen and Immunoadjuvant Model

Whether the fusion protein can stimulate the production of specific killer T cells (CTL, CD3+ CD8+OT-1+CD44+ T cells) and memory effector T cells (Tem, CD3+ CD8+CD44+CD62L− T cells) in a humanized mouse was verified in hCD40×h4-1BB KI humanized mouse (commercially available from BIOCYTOGEN) by using OVA (Sigma-Aldrich, A5503) as a model antigen and TLR3 agonist poly IC:LC (Sigma-Aldrich, P1530) as an immunoadjuvant.

The drugs were intraperitoneally (i.p.) administrated on days 1 and 6. The groups were the group of normal saline, the group of the model antigen and immunoadjuvant and the group of the fusion protein together with the model antigen and immunoadjuvant (the model antigen and immunoadjuvant were mixed and administrated and the fusion protein was administrated alone). Normal saline (10 μl/g body weight) was administrated to the group of normal saline. Mixed drug (10 μl/g body weight) of OVA (12.5 mg/kg) and polyIC:LC (1.25 mg/kg) was administrated to the group of the model antigen and immunoadjuvant. The fusion protein (5 mg/kg) and the mixed drug (10 μl/g body weight) of OVA (12.5 mg/kg) and polyIC:LC (1.25 mg/kg) were administrated to the group of the fusion protein together with the model antigen and immunoadjuvant. Heparin anticoagulated whole blood was taken on days 6 and 12 after a first administration, and the percentages of CTL and Tem were detected. Specifically, peripheral blood was taken with 100 μL/tube and OT-1 tetramer (MBL, TS-5001-1c) was added and incubated at 4° C. for 30 min. Then, anti-mouse CD45 (Biolegend, 103126), anti-mouse CD3e (Biolegend, 100234), anti-mouse CD8a (MBL, D271-4), anti-mouse CD44 (Biolegend, 103030) and anti-mouse CD62L (Biolegend, 104408) were added and incubated at 4° C. for 30 min. Erythrocyte lysate (Gibco, 11814389001) was added at 1 mL/tube, and the red blood cells were lysed for 5 min at room temperature protected from light, washed once with 1×PBS at 3 mL/tube, and resuspended with 1×PBS at 300 μL/tube, and flow cytometry was used to detect the specific killer T cells (CTL) and memory effector T cells (Tem).

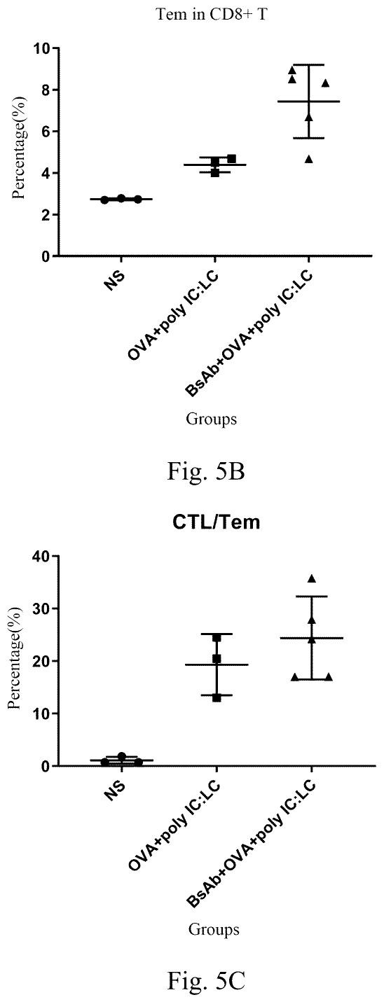

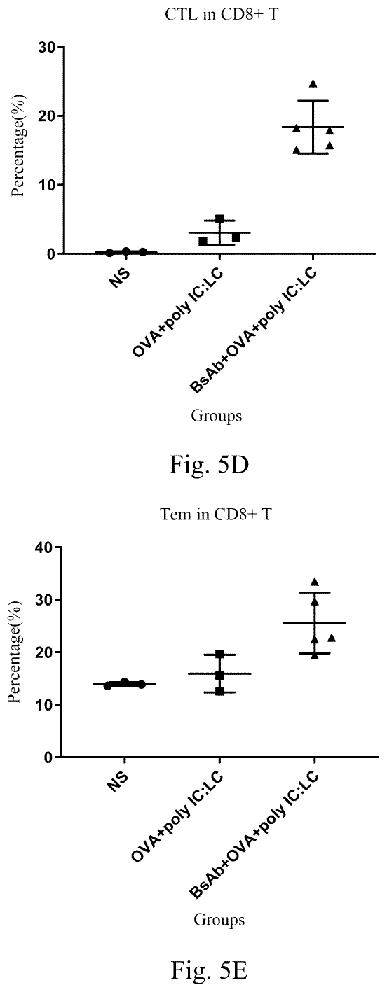

On day 6 after the first administration, the fusion protein can stimulate the production of specific killer T cells (CTL) and memory effector T cells (Tem) in humanized mice, with an increase in CTL of about 2% (FIG. 5A), Tem of about 7% (FIG. 5B), and CTL/Tem of about 25% (FIG. 5C).

On day 12 after the first administration, the fusion protein can stimulate the production of specific killer T cells (CTL) and memory effector T cells (Tem) in humanized mice, with an increase in CTL of about 20% (FIG. 5D), Tem of about 25% (FIG. 5E), and CTL/Tem of about 70% (FIG. 5F).

### Example 6. Inhibition of Growth of B16-OVA Subcutaneously Transplanted Tumor by Fusion Protein, Together with Model Antigen and Immunoadjuvant Model in Mice

hCD40×h4-1BB KI mice were used to verify whether the fusion protein of the present application, together with the model antigen and immunoadjuvant model, could inhibit the growth of subcutaneously transplanted tumor B16-OVA in the mice.

Establishment of Subcutaneous Transplant Tumor Model. Tumor cells (B16-OVA cells, donated by Immunoah Therapeutics, In) were injected subcutaneously with a concentration of 2×10⁵ cells/animal.

On day 7 after tumor injection, the length and width of the tumor were measured and the tumor volume was calculated according to v=ab²/2 (a was the length of the tumor and b was the width of the tumor). When the tumor volume reached 80˜120 mm³, the mice were randomized into groups. The groups were the group of normal saline, the group of the model antigen and the immunoadjuvant and the group of the fusion protein together with the model antigen and immunoadjuvant (the model antigen and immunoadjuvant were mixed and administrated and the fusion protein was administrated alone). Normal saline (10 μl/g body weight) was administrated to the group of normal saline. Mixed drug (10 μl/g body weight) of OVA (12.5 mg/kg) and polyIC:LC (1.25 mg/kg) was administrated to the group of the model antigen and the immunoadjuvant. The fusion protein (5.5 mg/kg) and the mixed drug (10 μl/g body weight) of OVA (12.5 mg/kg) and polyIC:LC (1.25 mg/kg) were administrated to the group of the fusion protein together with the model antigen and immunoadjuvant. The administration was carried out intraperitoneally, once every six days for a total of two administrations. Tumor volumes and body weights of mice were measured every 2-3 days. The tumor volume data were statistically analyzed at the end of the experiment.

After the mice were sacrificed, heparin anticoagulated whole blood and tumor tissues were taken and the percentages of specific killer T cells (CTL) and memory effector T cells (Tem) were detected. Meanwhile, hepatotoxicity-related indicators were detected after obtaining mouse serum.

Specifically, a suspension of peripheral blood and tumor tissues was taken with 100 μL/tube and OT-1 tetramer (MBL, TS-5001-1c) was added and incubated at 4° C. for 30 min. Then, anti-mouse CD45 (Biolegend, 103126), anti-mouse CD3e (Biolegend, 100234), anti-mouse CD8a (MBL, D271-4), anti-mouse CD44 (Biolegend, 103030) and anti-mouse CD62L (Biolegend, 104408) were added and incubated at 4° C. for 30 min. Erythrocyte lysate (Gibco, 11814389001) was added at 1 mL/tube, and the red blood cells were lysed for 5 min at room temperature protected from light, washed once with 1×PBS at 3 mL/tube, and resuspended with 1×PBS at 300 μL/tube, and flow cytometry was used to detect the specific killer T cells (CTL) and memory effector T cells (Tem).

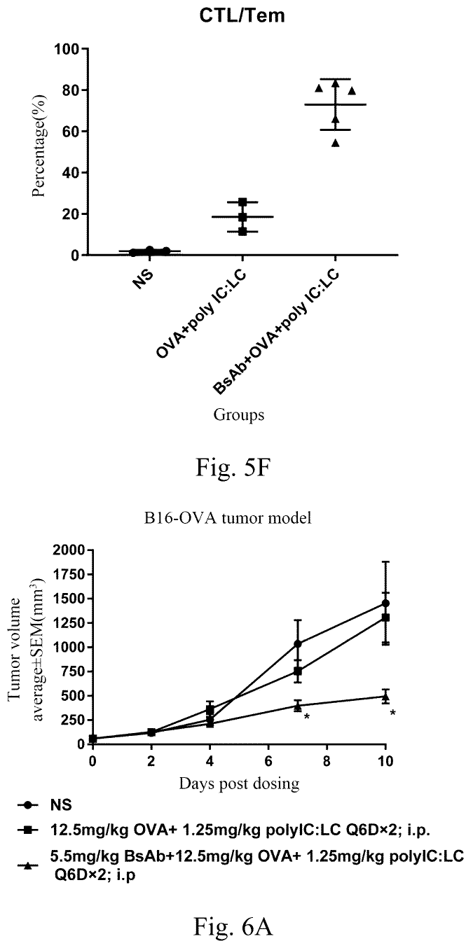

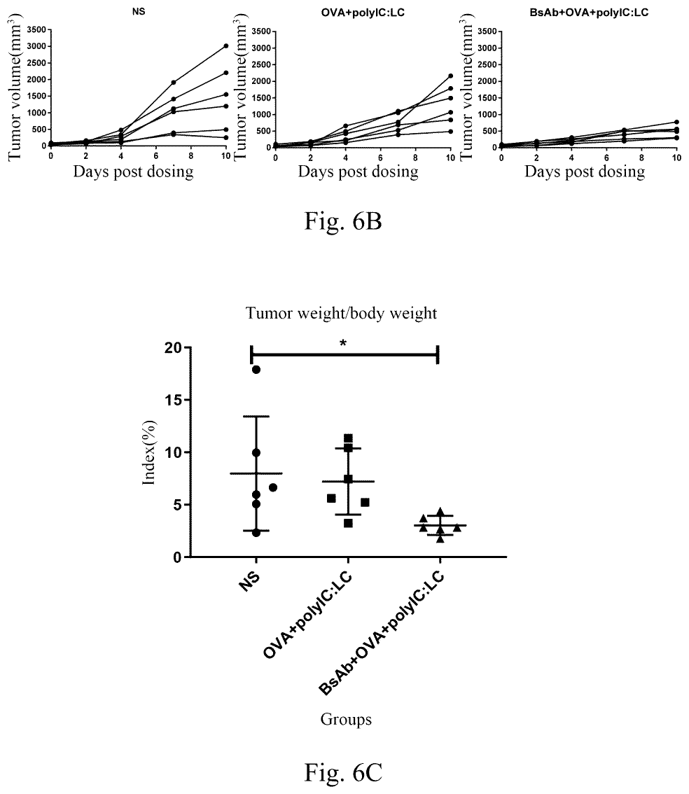

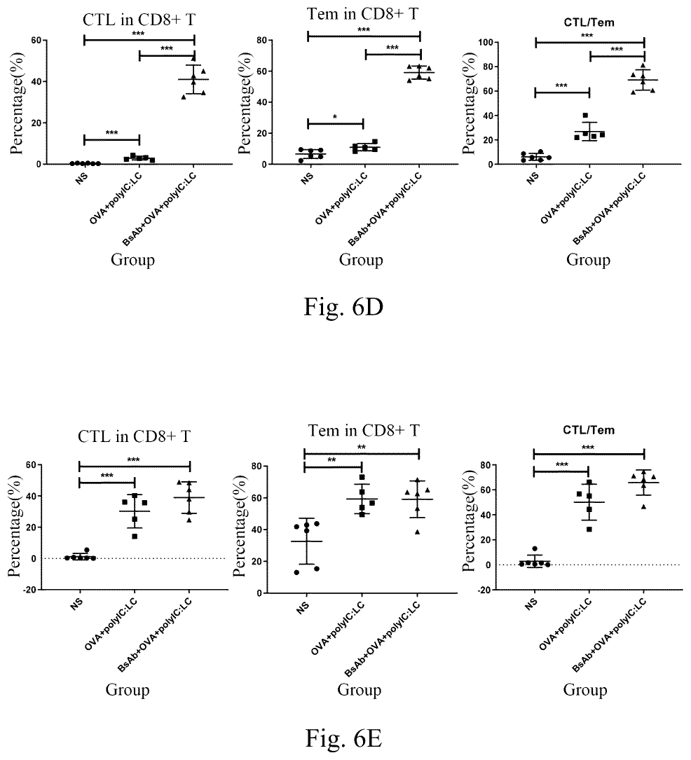

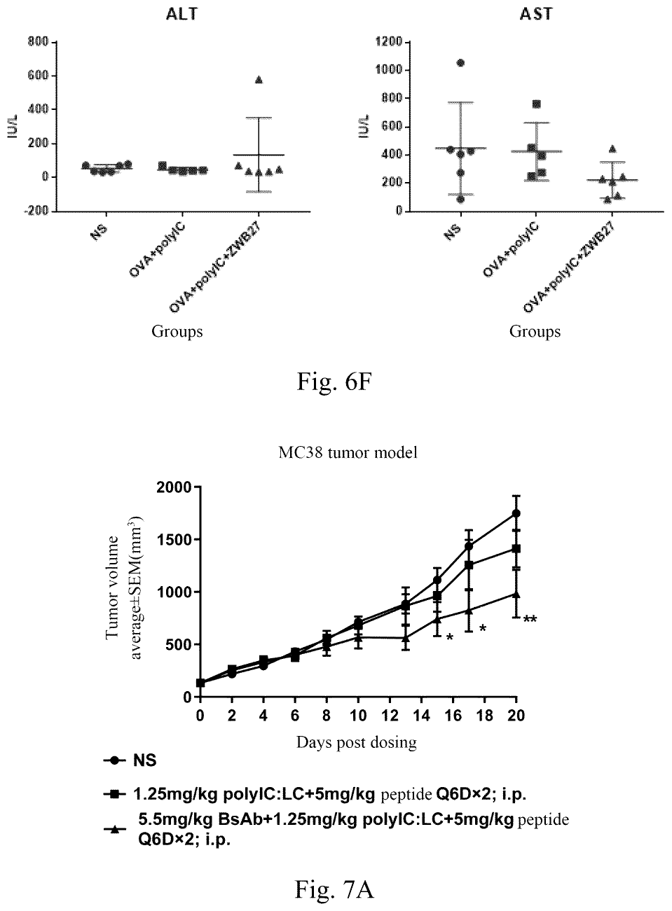

The results showed that the fusion protein, together with the model antigen and immunoadjuvant model could inhibit the increase of the volume of the B16-OVA tumor, and the inhibition rate reached 60%. Each point in the figure represented an assay from an individual animal. At the end of the experiment, the fusion protein together with the model antigen and immunoadjuvant model could significantly stimulate the production of CTL and Tem (FIGS. 6D and 6E). The fusion protein was not hepatotoxic to mice at a dose of 5.5 mg/kg (FIG. 6F).

### Example 7. Inhibition of Growth of MC38 Subcutaneously Transplanted Tumor by Fusion Protein, Together with MC38 Tumor Antigen and Immunoadjuvant Model in Mice

hCD40×h4-1BB KI mice were used to verify whether the fusion protein of the present application, together with the tumor antigen and the immunoadjuvant model, could inhibit the growth of subcutaneously transplanted tumor B16-OVA in the mice.

Establishment of Subcutaneous Transplant Tumor Model. Tumor cells (MC38cells, donated by Immunoah Therapeutics, In) were injected subcutaneously with a concentration of 4×10⁵ cells/animal.

On days 7-9 after tumor injection, the length and width of the tumor were measured and the tumor volume was calculated according to v=ab²/2 (a was the length of the tumor and b was the width of the tumor). When the tumor volume reached 80˜120 mm³, the mice were randomized into groups. The groups were the group of normal saline, the group of the MC38 tumor antigen and the immunoadjuvant and the group of the fusion protein together with the the MC38 tumor antigen and the immunoadjuvant (the MC38 tumor antigen and immunoadjuvant were mixed and administrated and the fusion protein was administrated alone). Normal saline (10 μl/g body weight) was administrated to the group of normal saline. Mixed drug (10 μl/g body weight) of the MC38 antigen tumor (5 mg/kg) and polyIC:LC (1.25 mg/kg) was administrated to the group of the model antigen and the immunoadjuvant. The fusion protein (5.5 mg/kg) and the mixed drug (10 μl/g body weight) of OVA (12.5 mg/kg) and polyIC:LC (1.25 mg/kg) were administrated to the group of the fusion protein together with the MC38 tumor antigen (5 mg/kg) and the immunoadjuvant. The administration was carried out intraperitoneally, once every six days for a total of two administrations. Tumor volumes and body weights of mice were measured every 2-3 days. The tumor volume data were statistically analyzed at the end of the experiment.

After the mice were sacrificed, heparin anticoagulated whole blood and tumor tissues were taken and the percentages of specific killer T cells (CTL) and memory effector T cells (Tem) were detected.

Specifically, a suspension of peripheral blood and tumor tissues was taken with 100 μL/tube and Adpgk tetramer (MBL, TB-5113-1) was added and incubated at 4° C. for 30 min. Then, anti-mouse CD45 (Biolegend, 103126), anti-mouse CD3e (Biolegend, 100234), anti-mouse CD8a (MBL, D271-4), anti-mouse CD44 (Biolegend, 103030) and anti-mouse CD62L (Biolegend, 104408) were added and incubated at 4° C. for 30 min. Erythrocyte lysate (Gibco, 11814389001) was added at 1 mL/tube, and the red blood cells were lysed for 5 min at room temperature protected from light, washed once with 1×PBS at 3 mL/tube, and resuspended with 1×PBS at 300 μL/tube, and flow cytometry was used to detect the specific killer T cells (CTL) and memory effector T cells (Tem).

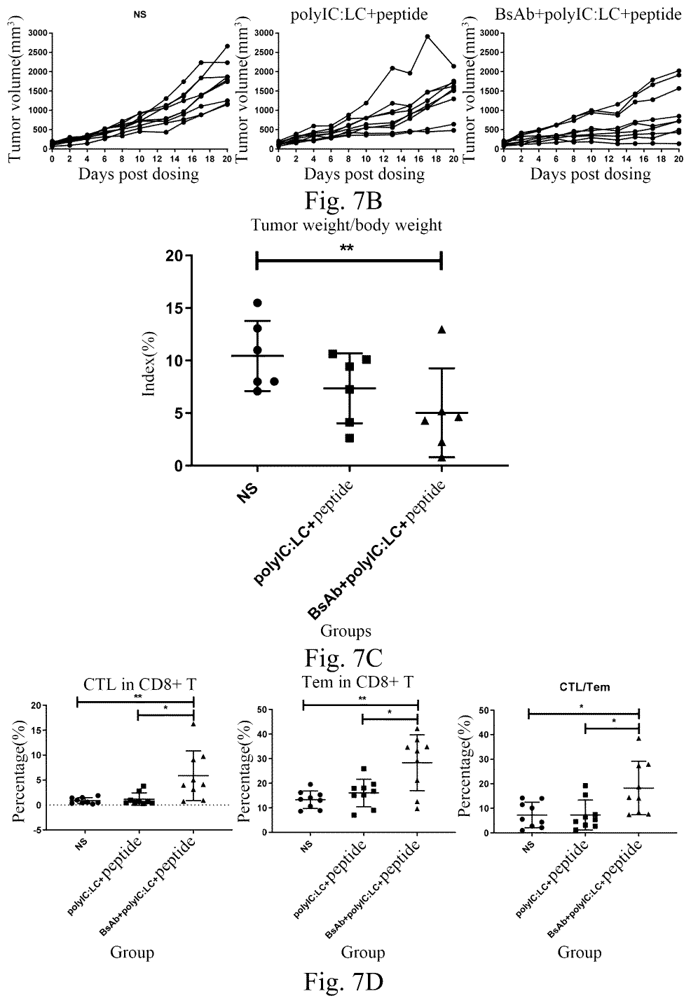

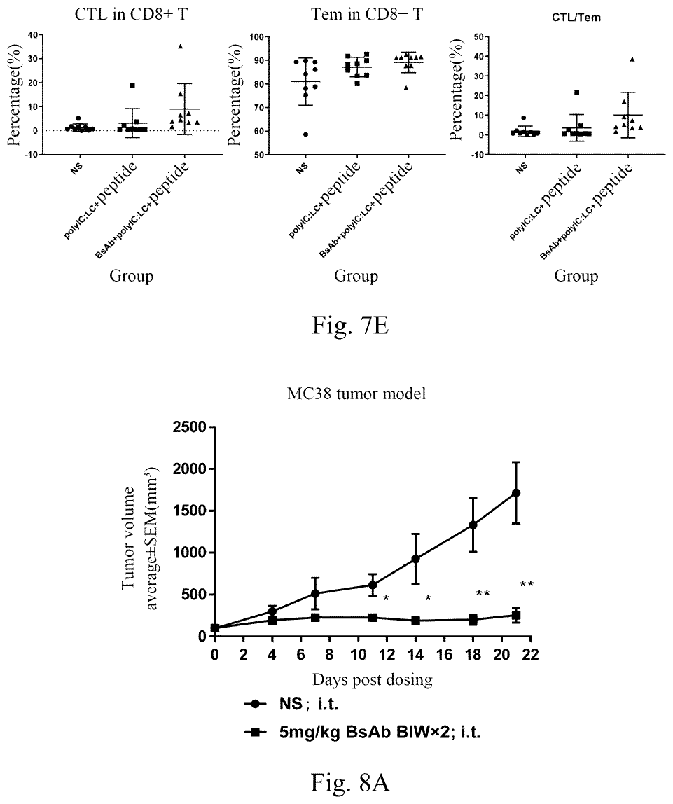

The results showed that the fusion protein, together with the tumor antigen and the immunoadjuvant model could inhibit the increase of the volume of the MC38 tumor, and the inhibition rate reached about 40%. Each point in the figure represented an assay from an individual animal. At the end of the experiment, the fusion protein together with the MC38 tumor antigen and the immunoadjuvant model could significantly stimulate the production of CTL and Tem (FIGS. 7D and 7E).

### Example 8. Inhibition of Growth of MC38 Subcutaneously Transplanted Tumor in Mice by Intratumoral Administration of Fusion Protein

hCD40×h4-1BB KI mice were used to verify whether intratumoral administration of the fusion protein of the present application could inhibit the growth of subcutaneously transplanted tumor MC38 in the mice.

Establishment of Subcutaneous Transplant Tumor Model. Tumor cells (MC38 cells) were injected subcutaneously with a concentration of 4×10⁵ cells/animal. On days 7-9 after tumor injection, the length and width of the tumor were measured and the tumor volume was calculated according to v=ab²/2 (a was the length of the tumor and b was the width of the tumor). When the tumor volume reached 80˜120 mm³, the mice were randomized into groups. The groups were the group of normal saline, and the group of fusion protein. Normal saline (2 μl/g body weight) was administrated to the group of normal saline. The fusion protein (5.0 mg/kg, 2 μl/g body weight) was administrated to the group of fusion protein. The administration was carried out intratumorally, twice every week for a total of four administrations. Tumor volumes and body weights of mice were measured every 2-3 days. The tumor volume data were statistically analyzed at the end of the experiment.

After the mice were sacrificed, heparin anticoagulated whole blood and tumor tissues were taken and the percentages of memory effector T cells (Tem) and regulatory T cells (Tregs) were detected. Meanwhile, hepatotoxicity-related indicators were detected after obtaining mouse serum.

Specifically, a suspension of peripheral blood and tumor tissues was taken with 100 μL/tube. Anti-mouse CD45 (Biolegend, 130112), anti-mouse CD3e (Biolegend, 100234), anti-mouse CD8a (Biolegend, 100714), anti-mouse CD44 (Biolegend, 103022) and anti-mouse CD62L (Biolegend, 104408) were added and incubated at 4° C. for 30 min. Erythrocyte lysate (Gibco, 11814389001) was added at 1 mL/tube, and the red blood cells were lysed for 5 min at room temperature protected from light, washed once with 1×PBS at 3 mL/tube, and resuspended with 1×PBS at 300 μL/tube, and flow cytometry was used to detect the memory effector T cells (Tem).

A suspension of peripheral blood and tumor tissues was taken with 100 μL/tube and anti-mouse CD45 (Biolegend, 103132), anti-mouse CD3e (Biolegend, 100204), anti-mouse CD8a (Biolegend, 100714), anti-mouse CD44 (Biolegend, 100449) and anti-mouse CD25 (Biolegend, 102016) were added and incubated at 4° C. for 30 min. Erythrocyte lysate (Gibco, 11814389001) was added at 1 mL/tube, and the red blood cells were lysed for 5 min at room temperature protected from light, and washed once with 1×PBS at 3 mL/tube. The supernatant was discarded and 1 mL Fix/Perm liquid (Invitrogen, 88-8824-00) was added. The mixture was incubated for 1h at room temperature protected from light, washed once with 2 mL of 1× Perm buffer (Invitrogen, 00-833-56). The supernatant was discarded. Anti-mouse CD25 (Biolegend, 320008) was added and incubated for 30 min at room temperature protected from light, washed once with 2 mL of 1× Perm buffer, and the supernatant was discarded. The cells were suspended with 1× Perm buffer at 300 μL/tube, and flow cytometry was used to detect the regulatory T cells (Tregs).

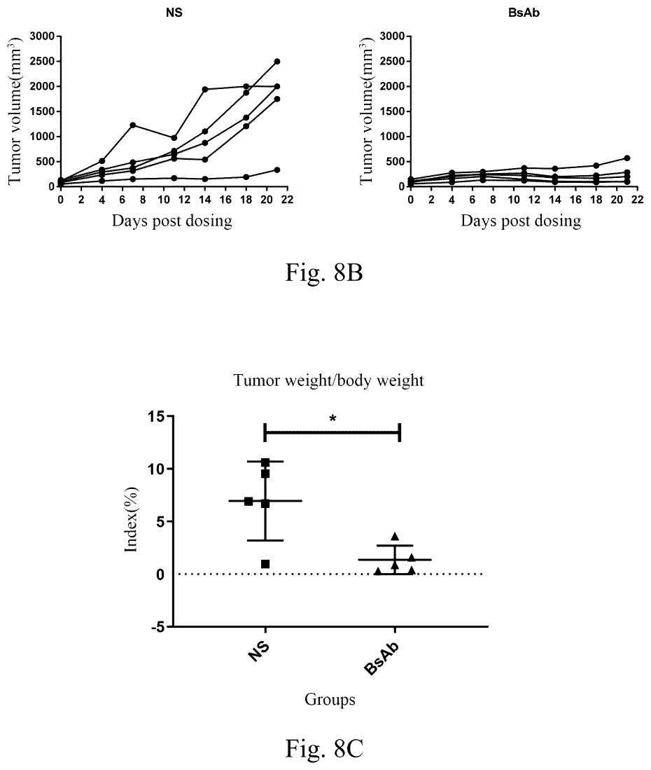

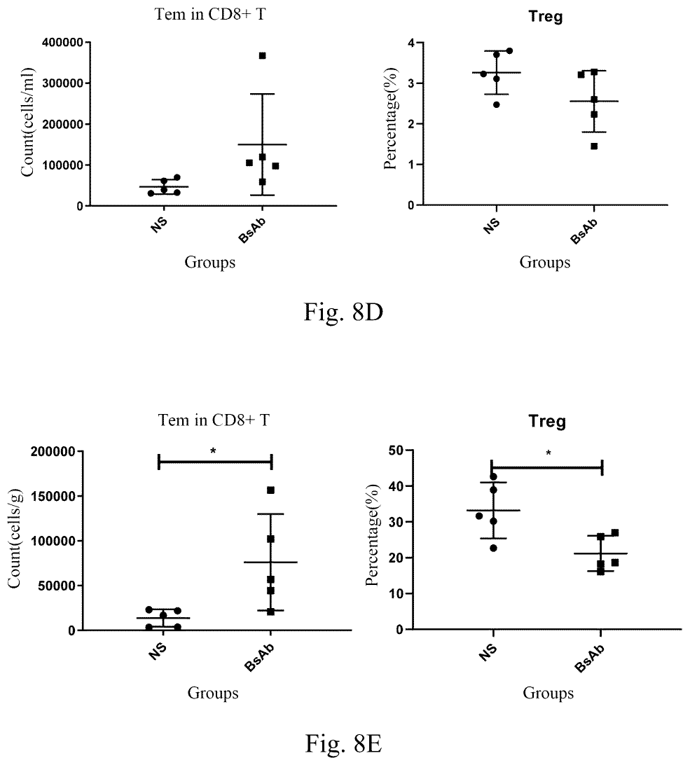

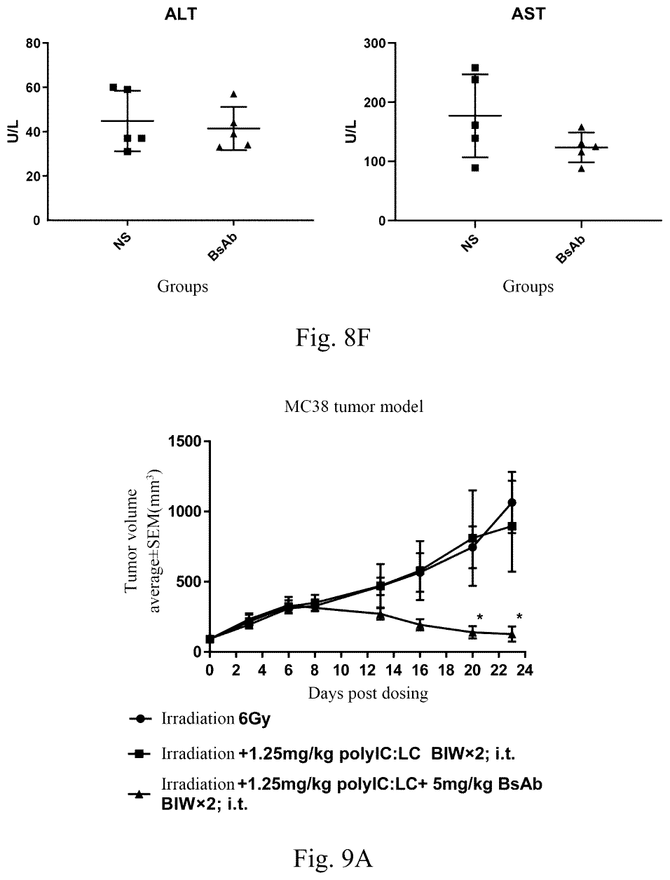

The results showed that the fusion protein could inhibit the increase of the volume of the MC38tumor, and the inhibition rate reached 85%. Each point in the figure represented an assay from an individual animal. At the end of the experiment, the fusion protein could stimulate the production of CTL (FIG. 8D) and reduce the proportion of Tregs. The fusion protein was not hepatotoxic to mice at a dose of 5.5 mg/kg (FIG. 8F).

### Example 9. Inhibition of Growth of MC38 Subcutaneously Transplanted Tumor in Mice by Intratumoral Administration of Fusion Protein in Combination with Radiotherapy and Immunoadjuvant Model

hCD40×h4-1BB KI mice were used to verify whether intratumoral administration of the fusion protein of the present application in combination with radiotherapy and immunoadjuvant model could inhibit the growth of subcutaneously transplanted tumor MC38 in the mice.

Establishment of Subcutaneous Transplant Tumor Model. Tumor cells (MC38 cells) were injected subcutaneously with a concentration of 4×10⁵ cells/animal. On days 7-9 after tumor injection, the length and width of the tumor were measured and the tumor volume was calculated according to v=ab²/2 (a was the length of the tumor and b was the width of the tumor). When the tumor volume reached 80˜120 mm3, the mice were randomized into groups. The groups were the group of normal saline, the group of radiotherapy and immunoadjuvant and the group of fusion protein and radiotherapy and immunoadjuvant (the immunoadjuvant and fusion protein were administrated separately). Normal saline (2 μl/g body weight) was administrated to the group of normal saline. polyIC:LC (1.25 mg/kg, 10 μl/g body weight) was administrated to the group of immunoadjuvant. The fusion protein (5.0 mg/kg, 2 μl/g body weight) and polyIC:LC (1.25 mg/kg, 2 μl/g body weight) were administrated to the group of fusion protein and immunoadjuvant. After irradiation with cobalt source of 6Gy, the administration was carried out intratumorally, twice every six weeks for a total of four administrations. Tumor volumes and body weights of mice were measured every 2-3 days. The tumor volume data were statistically analyzed at the end of the experiment.

After the mice were sacrificed, heparin anticoagulated whole blood and tumor tissues were taken and the percentages of memory effector T cells (Tem) and regulatory T cells (Tregs) were detected. Meanwhile, hepatotoxicity-related indicators were detected after obtaining mouse serum.

Specifically, a suspension of peripheral blood and tumor tissues was taken with 100 μL/tube. Anti-mouse CD45 (Biolegend, 130112), anti-mouse CD3e (Biolegend, 100234), anti-mouse CD8a (Biolegend, 100714), anti-mouse CD44 (Biolegend, 103022) and anti-mouse CD62L (Biolegend, 104408) were added and incubated at 4° C. for 30 min. Erythrocyte lysate (Gibco, 11814389001) was added at 1 mL/tube, and the red blood cells were lysed for 5 min at room temperature protected from light, washed once with 1×PBS at 3 mL/tube, and resuspended with 1×PBS at 300 μL/tube, and flow cytometry was used to detect the memory effector T cells (Tem).

A suspension of peripheral blood and tumor tissues was taken with 100 μL/tube and anti-mouse CD45 (Biolegend, 103132), anti-mouse CD3e (Biolegend, 100204), anti-mouse CD8a (Biolegend, 100714), anti-mouse CD44 (Biolegend, 100449) and anti-mouse CD25 (Biolegend, 102016) were added and incubated at 4° C. for 30 min. Erythrocyte lysate (Gibco, 11814389001) was added at 1 mL/tube, and the red blood cells were lysed for 5 min at room temperature protected from light, and washed once with 1×PBS at 3 mL/tube. The supernatant was discarded and 1 mL Fix/Perm liquid (Invitrogen, 88-8824-00) was added. The mixture was incubated for 1h at room temperature protected from light, washed once with 2 mL of 1× Perm buffer (Invitrogen, 00-833-56). The supernatant was discarded. Anti-mouse CD25 (Biolegend, 320008) was added and incubated for 30 min at room temperature protected from light, washed once with 2 mL of 1× Perm buffer, and the supernatant was discarded. The cells were suspended with 1× Perm buffer at 300 μL/tube, and flow cytometry was used to detect the regulatory T cells (Tregs).

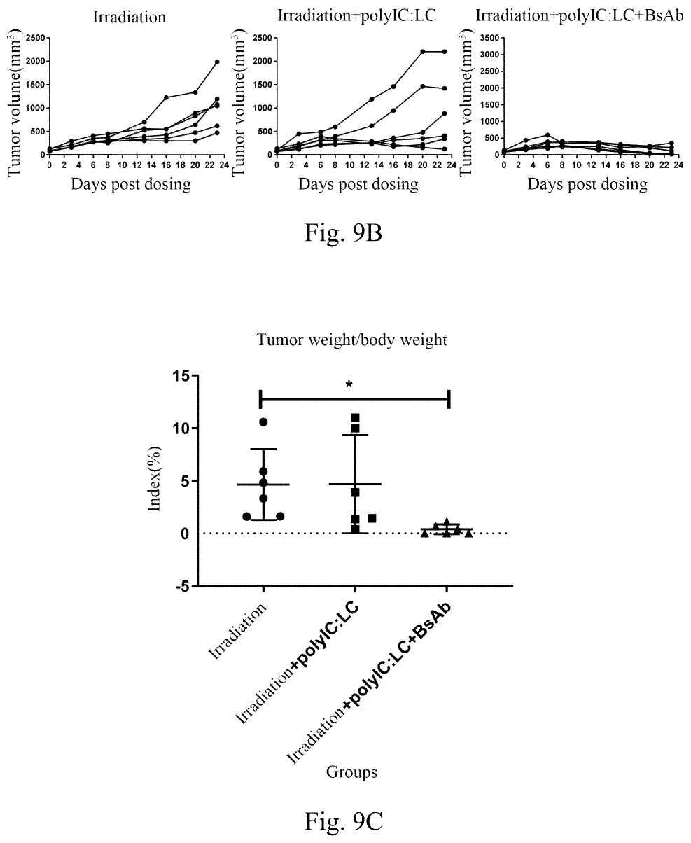

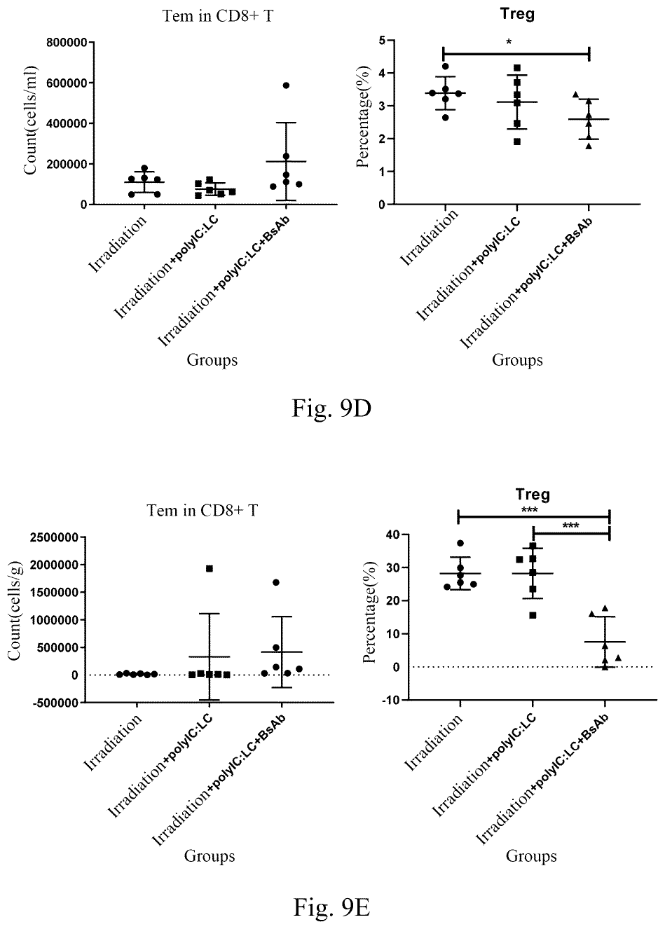

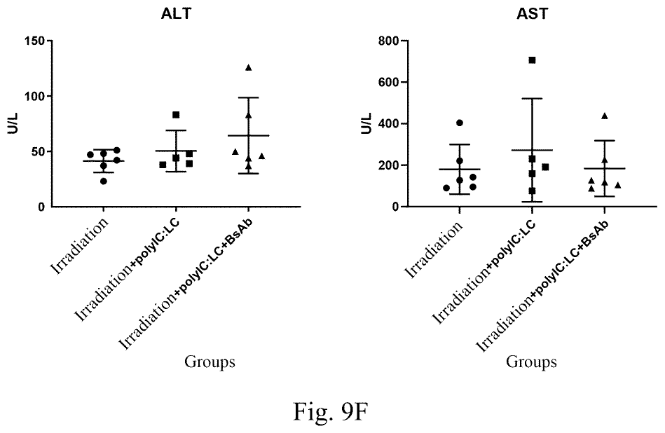

The results showed that intratumoral administration of the fusion protein in combination with the radiotherapy and immunoadjuvant could inhibit the increase of the volume of the MC38tumor, and the inhibition rate reached 86%. Each point in the figure represented an assay from an individual animal. At the end of the experiment, intratumoral administration of the fusion protein in combination with the radiotherapy and immunoadjuvant could stimulate the production of CTL (FIG. 9D) and reduce the proportion of Tregs. The fusion protein was not hepatotoxic to mice at a dose of 5.5 mg/kg (FIG. 9F).

It is understood that although the involved inventions are described in the above particular form in the present application, these inventions are not limited to the particular contents described by these specific forms. It will be apparent to those skilled in the art that various equivalents can be made to the technical features contained in the inventions referred to therein without departing from the spirit of the inventions described by the present application, which variations are intended to be within the scope of the inventions.

1. A fusion protein comprising:
a) a Fab fragment specifically binding to 4-lBB molecules;
b) a first CD40L specifically binding to CD40 molecules, the N-terminus of the first CD40L being linked to the C-terminus of the light chain or heavy chain of the Fab fragment by a first peptide linker, and
c) a second CD40L specifically binding to CD40 molecules, the N-terminus of the second CD40L being linked to the C-terminus of the heavy chain or light chain of the Fab fragment by a second peptide linker,
wherein only one disulfide bond can be formed between the first peptide linker and the second peptide linker, and each of the peptide linkers is independently selected from the group consisting of a peptide linker comprising any of the sequences as set forth in SEQ ID NOs: 24-25, wherein X represents any amino acids other than Cys, or is absent.

2. The fusion protein of claim 1, wherein the fusion protein further comprises d) a third CD40L specifically binding to CD40 molecules, the N-terminus of the third CD40L being linked to the C-terminus of the first CD40L or the second CD40L by a third peptide linker.

3. The fusion protein of claim 1, wherein the 4-1 BB molecule and the 0D40 molecule are independently derived from a mammal, preferably a non-human primate or human.

4. The fusion protein of claim 1, wherein the fusion protein specifically binds to the 0D40 molecule and the 4-1 BB molecule with an affinity of at least 1×10⁻⁸, and the fusion protein has the function of 0D40 agonist and 4-1 BB agonist and is capable of inducing dendritic cell maturation and/or T cell activation.

5. A nucleic acid encoding the fusion protein of claim 1.

6. An expression vector comprising the nucleic acid of claim 5.

7. A host cell comprising the nucleic acid of claim 5, wherein the host cell is a mammalian cell selected from the group consisting of a CHO cell, a NS0 cell, a SP2/0 cell, a HEK293 cell, a COS cell and a PER.C6 cell.

8. (canceled)

9. A pharmaceutical composition comprising the fusion protein of claim 1, and a pharmaceutically acceptable carrier.

10. (canceled)

11. (canceled)

12. A method for treating, ameliorating or preventing a tumor, an immune-related disease or an infectious disease in a subject, comprising administering to the subject the fusion protein of claim 1.

13. The method of claim 12, wherein the tumor is selected from the group consisting of lung cancer, mesocorticoma, colorectal cancer, bladder cancer, leukemia, breast cancer, gastric cancer, adenocarcinoma of the gastro-oesophageal junction, non-Hodgkin's lymphoma, Hodgkin's lymphoma, anaplastic large cell lymphoma, head and neck cancer, malignant glioma, renal cancer, melanoma, prostate cancer, bone cancer, pancreatic cancer, sarcoma, liver cancer, skin squamous cell carcinoma, cervical cancer, nasal pharynx cancer, endometrial cancer, or metastatic cancer of any of the above tumors; or the immune-related disease is selected from the group consisting of systemic lupus erythematosus, rheumatoid arthritis, systemic vasculitis and autoimmune hemolytic anemia; or the infectious disease is selected from the group consisting of influenza, colitis, HPV infection, hepatitis B, rabies, syphilis and AIDS.

14. The fusion protein of claim 1, wherein the first peptide linker and/or the second peptide linker are a hinge region of a native antibody, and wherein a mutation that retains only one cysteine is made to the hinge region.

15. The fusion protein of claim 1, wherein the first peptide linker and/or the second peptide linker are the IgG1 hinge region with C239 being deleted or substituted, or the IgG1 hinge region with C239 being deleted or substituted and the hinge region D234-S252 being inverted.

16. The fusion protein of claim 2, wherein the fusion protein further comprises e) FcBP, wherein the FcBP is linked to the C-terminus of any one or more of the first CD40L, the second CD40L, and the third CD40L.

17. The fusion protein of claim 1, wherein a fourth peptide linker and a fifth peptide linker are respectively linked between the first peptide linker and the first CD40L, and between the second peptide linker and the second CD40L.

18. The fusion protein of claim 1, wherein the third peptide linker, the fourth peptide linker, and the fifth peptide linker each independently comprise one or more of the sequence as set forth in SEQ ID NO: 5, the sequence as set forth in SEQ ID NO:6, the sequence as set forth in a plurality of SEQ ID NOs: 5 linked in series, and a plurality of SEQ ID NOs: 6 linked in series.

19. The fusion protein of claim 1, wherein the 4-1 BB molecule and the CD40 molecule are independently derived from a non-human primate or human.

20. The fusion protein of claim 1, wherein the Fab fragment comprises HCDR1 as set forth in SEQ ID NO: 7, HCDR2 as set forth in SEQ ID NO: 8, and HCDR3 as set forth in SEQ ID NO: 9, and LCDR1 as set forth in SEQ ID NO: 10, LCDR2 as set forth in SEQ ID NO: 11, and LCDR3 as set forth in SEQ ID NO: 12.

21. The fusion protein of claim 20, wherein the Fab fragment comprises the heavy chain variable region as set forth in SEQ ID NO: 13, and the light chain variable region as set forth in SEQ ID NO: 14.

22. The fusion protein of claim 2, wherein the first CD40L, second CD40L and third CD40L each independently comprise any of SEQ ID NOs: 1-4.

23. The fusion protein of claim 16, wherein the FcBP comprises the sequence as set forth in SEQ ID NO: 15.


SEQUENCE LISTING (25 sequences)

SEQ ID NO: 1 - protein, 149 aa, Homo sapiens
MQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 2 - protein, 146 aa, Homo sapiens
GDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 3 - protein, 143 aa, Homo sapiens
NPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 4 - protein, 141 aa, Homo sapiens
QIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 5 - protein, 9 aa, synthetic construct
GGGGSGGGS

SEQ ID NO: 6 - protein, 5 aa, synthetic construct
GGGGS

SEQ ID NO: 7 - protein, 5 aa, synthetic construct
SYWMD

SEQ ID NO: 8 - protein, 17 aa, synthetic construct
NIYPDSGGTNYAEKFKS

SEQ ID NO: 9 - protein, 12 aa, synthetic construct
EEALGGYYELTY

SEQ ID NO: 10 - protein, 15 aa, synthetic construct
RASQSVSTSSYSYMH

SEQ ID NO: 11 - protein, 7 aa, synthetic construct
YASNLES

SEQ ID NO: 12 - protein, 9 aa, synthetic construct
QHSWEIPYT

SEQ ID NO: 13 - protein, 121 aa, synthetic construct
EVQLVQSGAEVKKPGASVKVSCKASGYTFTSYWMDWVRQAPGQGLEWMGNIYPDSGGTNYAEKFKSRVTLTVDTSISTAYMELSRLRSDDTAVYYCAREEALGGYYELTYWGQGTLVTVSS

SEQ ID NO: 14 - protein, 111 aa, synthetic construct
DIVMTQSPDSLAVSLGERATINCRASQSVSTSSYSYMHWYQQKPGQPPKLLIKYASNLESGVPDRFSGSGSGTDFTLTISSLQAEDVAVYYCQHSWEIPYTFGGGTKVEIK

SEQ ID NO: 15 - DNA, 48 bp, synthetic construct
  source 1..48: mol_type = other DNA
cagagatttgtgaccggccacttcggcggcctgtatcctgctaatgga

SEQ ID NO: 16 - DNA, 53 bp, synthetic construct
  source 1..53: mol_type = other DNA
tgtggctgagaggtgccagatgtgaagtgcagctggttcagtctggcgccgaa

SEQ ID NO: 17 - DNA, 48 bp, synthetic construct
  source 1..48: mol_type = other DNA
cgcaatttgaggattctgatcaccgctgccgccgccgcctgacggtcc

SEQ ID NO: 18 - DNA, 46 bp, synthetic construct
  source 1..46: mol_type = other DNA
gaccgtcaggcggcggcggcagcggtgatcagaatcctcaaattgc

SEQ ID NO: 19 - DNA, 55 bp, synthetic construct
  source 1..55: mol_type = other DNA
caatttgaggattctgatcaccagagcctccaccagagcctccacccccgagttt

SEQ ID NO: 20 - DNA, 54 bp, synthetic construct
  source 1..54: mol_type = other DNA
caaactcgggggtggaggctctggtggaggctctggtgatcagaatcctcaaat

SEQ ID NO: 21 - DNA, 48 bp, synthetic construct
  source 1..48: mol_type = other DNA
tgattatgatcaatgaattcctagagtttgagtaagccaaaggacgtg

SEQ ID NO: 22 - DNA, 47 bp, synthetic construct
  source 1..47: mol_type = other DNA
acgtcctttggcttactcaaactccagagatttgtgaccggccactt

SEQ ID NO: 23 - DNA, 44 bp, synthetic construct
  source 1..44: mol_type = other DNA
ctgattatgatcaatgaattctcatccattagcaggatacaggc

SEQ ID NO: 24 - protein, 8 aa, synthetic construct
XPPCPAPE

SEQ ID NO: 25 - protein, 8 aa, synthetic construct
EPAPCPPX